(19) United States
(12) Patent Application Publication
(10) Pub. No.: US 2006/0099710 A1
(43) Pub. Date: May 11, 2006

(54) Vector for improved in vivo production of proteins

(76) Inventors: Mark I. Donnelly, Warrenville, IL (US); Andrzej Joachimiak, Bolingbrook, IL (US)

Correspondence Address: BARNES & THORNBURG, LLP, P.O. BOX 2786, CHICAGO, IL, 60690-2786, US

(21) Appl. No.: 11/196,366

(22) Filed: Aug. 3, 2005

Related U.S. Application Data
(60) Provisional application No. 60/626,800, filed on Nov. 10, 2004.

(51) Int. Cl. C12N 15/86; C12N 5/06; C12N 5/04; C07H 21/02

(52) U.S. Cl. 435/456; 536/23.1; 435/468; 435/325; 435/348; 435/419; 435/252.3

(57) ABSTRACT

A vector designed to include two tags wherein the first tag improves protein expression, folding and solubility, and the second tag promotes affinity purification, and two distinct recognition sequences for highly specific proteases. Uses of the vector include in vivo protein production.

### CROSS REFERENCE TO RELATED APPLICATION

This application claims priority to U.S. Provisional Application, Ser. No. 60/626,800, filed Nov. 10, 2004, the disclosure of which is fully incorporated herein by reference.

### GOVERNMENT RIGHTS

This invention was partially conceived under Contract No. W-31-109-ENG-38 between the U.S. Department of Energy and the University of Chicago representing Argonne National Laboratory. The U.S. Government may have certain rights in this invention.

### BACKGROUND

Many vectors have been made for expressing large amounts of proteins for protein purification, characterization and structural studies. Some vectors were designed for high throughput cloning and expression of target proteins, including vectors made at Argonne National Laboratory for the NIH-funded Structural Genomics Project. Some of these vectors incorporate attributes of known vectors, including the use of a polyhistidine affinity purification sequence (his-tag), a recognition sequence for the highly specific tobacco etch virus protease (TEV-site), the maltose binding protein (MBP), which improves the solubility of expressed proteins, and a sequence that allows ligation independent cloning (LIC) of target genes.

Maltose binding protein (MBP) is effective in enhancing the solubility of proteins over-expressed in E. coli when MBP is fused to the expressed protein. MBP is usually removed from the target protein after expression by a specific protease whose recognition sequence is inserted between MBP and the target protein. A suitable protease is the tobacco etch virus (TEV) protease, desirable for its high specificity and tolerance of various reaction conditions. In a variation of this approach, it is possible to co-express the protease with the MBP-target fusion, allowing in vivo processing to remove MBP.

Purification of target proteins is facilitated by attachment of affinity tags that bind selectively to particular materials. An example of a tag is the his-tag, which is a string of 6 to 10 consecutive histidine residues that binds strongly, yet reversibly, to metal ions chelated to certain resins, allowing purification by immobilized metal ion affinity chromatography (IMAC). The his-tags are usually followed by a protease recognition sequence that allows their removal after purification. This approach has been combined with MBP in various configurations, including the use of an N-terminally his-tagged MBP followed by the TEV protease recognition sequence.

A production vector, pMCSG7 (FIG. 1A), used by the Midwest Center for Structural Genomics, is based on the pET system of vectors. pMCSG7 encodes a leader sequence consisting of an N-terminal his₆-tag followed by a spacer and the tobacco etch virus (TEV) protease recognition sequence, and a LIC region based on a central SspI site. Hundreds of target proteins have been produced with this vector, leading to structural determination of over 100 proteins. High throughput protocols developed for purifying these proteins include a preliminary IMAC step followed by desalting, treatment with a his-tagged TEV protease and a second IMAC step to remove the protease and other proteins that bind the immobilized metal.

A vector designated pMCSG9 that includes some of the components mentioned above enhances the production of proteins for structural studies, but properties of the expressed fusion proteins often disrupt the normal high-throughput purification of the target proteins. In the case of pMCSG9, the expressed fusion proteins are a fusion of MBP and the target.

The vector pMCSG9 (FIG. 1B), a variant of pMCSG7, has the gene encoding MBP inserted between the his-tag and the TEV recognition sequence to improve the solubility of expressed proteins. The vector is effective in salvaging many proteins that are poorly soluble when expressed with only the his-tag. The effect of insertion of MBP into the leader sequence of 131 proteins on solubility was observed. Proteins that were insoluble (Solubility Score) or poorly soluble (Solubility Score 1), when expressed in pMCSG7 were produced from pMCSG9 with the leader his6-MBP-TEV site (FIGS. 2A-2B). However, integration of pMCSG9 into high-throughput purification protocols revealed serious limitations. First, not all proteins fused to MBP are rendered soluble by this association—many which are soluble while fused to MBP precipitate or aggregate when released by cleavage with TEV. Introduction of these targets into the purification pipeline generally fails to give sufficient material for crystallization trials, wasting time and resources. Second, with those proteins that remain soluble after TEV cleavage, the resulting his-tagged MBP interferes with semi-robotic purification protocols because the his₆-MPB binds less tightly to the IMAC resin than does its fusion with target protein, and fails to bind to the second IMAC column, which is intended to retain it. Because it is not retained, the standard protocols result in severe contamination of the final target protein (FIGS. 2C-2D). Additional, time-consuming steps or modifications of the standard protocols are needed to generate pure protein, for example, as needed for crystallization trials.

Target proteins are experimental proteins released from fusion proteins after TEV treatment. In these procedures, the expressed protein is first purified by immobilized-metal affinity chromatography (IMAC), which binds the his-tag. Normally, the his-tag is removed by treatment with TEV protease followed by dialysis and a second IMAC column that removes the his-tag and any host proteins that are bound to the first IMAC column, allowing the target protein to elute in pure form. However, the properties of the his-tagged MBP are incompatible with these high-throughput protocols. His-tagged MBP is too large to diffuse away during dialysis and binds less efficiently to the second IMAC column so that it is not fully retained, resulting in impure target protein. Therefore, additional or modified, more laborious steps are required to purify the target proteins from the his-tagged MBP, and the high-throughput process is disrupted.

An additional deficiency of previous MBP vectors is that sometimes the enhancement of target protein solubility is artificial. MBP often improves other proteins' folding and solubility, resulting in good yields of the soluble protein after MBP has been removed by treatment with TEV protease, but in some cases the target protein does not fold properly and is rendered “soluble” only by its fusion to the large, highly soluble MBP protein. Upon cleavage, the target protein remains insoluble and precipitates after separated from MBP. These “false positives” decrease the efficiency because they are processed through the labor intensive purification protocols, reducing the percentage of successful purifications and increasing the overall cost of the high-throughput purifications.

A protein expression and purification vector is desired that provides increased solubility and simpler downstream high throughput purification steps.

### SUMMARY

A nucleic acid molecule or a new expression vector design described herein eliminates the need for more laborious purification steps and restores high throughput processing of proteins expressed with maltose binding protein (MBP). The sequence of active elements of the vector also eliminates false positives, because after in vivo cleavage the expressed proteins that are not truly soluble, precipitate.

The new nucleic acid molecule includes:

(a) a first nucleotide sequence encoding a first tag (tag1);

(b) a second nucleotide sequence encoding a second tag (tag2);

(c) a third nucleotide sequence encoding a first recognition peptide sequence (site 1) for a first specific protease; and

(d) a fourth nucleotide sequence encoding a second recognition peptide sequence (site 2) for a second specific protease.

The nucleic acid molecule may further include a fifth nucleotide sequence encoding a target, which may be a protein or a peptide of interest.

A suitable tag1 may be a protein or a peptide that improves protein production expression, folding or solubility, for example, MBP. On the other hand, tag2 may be a protein or a peptide that promotes affinity purification, such as his₆. It is understood that tag2 may also be another marker gene such as fluorescent tag.

A suitable first specific protease and a second specific protease are distinct from one another and each may be selected from the proteases listed in TABLE I of the present disclosure. For example, the first specific protease may be a tobacco vein mottling virus (TVMV) protease, and the second specific protease may be a tobacco etch virus (TEV) protease. Accordingly, the corresponding site 1, which is cleaved by TVMV is designated tvmv, and the corresponding site 2, which is cleaved by TEV is designated tev. Many of the specific proteases are commercially available. TEV is commercially available (Invitrogen). TVMV may be coexpressed with a vector made by David S. Waugh and sold by Science Reagents, Inc. (El Cajon, Calif.).

The components of the nucleic acid molecule may be arranged so that the encoded peptide has the first recognition sequence (site1) positioned between the first tag (tag1) and the second tag (tag2), and the second recognition sequence (site2) positioned downstream or upstream of the second tag (tag2). For example, the peptide sequence may include tag1-site1-tag2-site2-target or target-site2-tag2-site1-tag1.

The new nucleic molecule may be constructed into an expression vector. The new vector may include other appropriate components that are known in the art such as T7 promoter and T7 terminator. The new vector differs from previous vectors at least in that it incorporates two tags, one to improve protein expression, folding and/or solubility, and the second to promote affinity purification, each followed by a distinct recognition sequence for a highly specific protease.

Alternative embodiments of the new vector include a nucleic molecule encoding the peptide sequence of N-helpertag-site1-markertag-site2-target, or a nucleic molecule encoding the peptide sequence of N-target-site 2-markertag-site1-helpertag, where N is the N-terminus of the peptide, helpertag is a protein or a peptide for improving protein expression, folding or solubility, site1 is a first recognition peptide sequence that is cleaved by a first specific protease, markertag is a peptide sequence used for purification or detection; site2 is a second recognition peptide sequence that is cleaved by a second specific protease, and target is a protein or peptide of interest.

A specific embodiment of a helpertag is MBP, of site1 is tvmv, of markertag is his₆, and of site2 is tev.

Specifically, the new vector, designated pMCSG19, produces a protein with the elements: N-MBP-tvmv-his₆-tev-target, where N is the N-terminus of the protein; MBP is a maltose binding protein tag; tvmv is the recognition sequence for the TVMV protease; his₆ is a six-histidine tag; tev is the recognition sequence for the TEV protease; and target is the desired proteins to be purified.

The above-described vectors and other vectors constructed in a similar fashion are useful for protein production. The improved method of protein production includes:

(a) expressing a target protein in a cell using a vector comprising a nucleotide sequence encoding a peptide sequence of N-helpertag-site1-markertag-site2-target, or a nucleotide sequence encoding a peptide sequence of N-target-site2-markertag-site1-helpertag, where N is N-terminus of the peptide, helpertag is a beneficial protein or peptide sequence for improving protein expression, folding or solubility, site1 is a recognition peptide sequence that is cleaved by a first specific enzyme, markertag is a peptide sequence used for purification, detection or other application, site2 is another recognition peptide sequence that is cleaved by a second specific enzyme, and target is a protein of interest. The method further includes:

(b) cleaving the encoded peptide at site1 with the first specific enzyme to produce a peptide of markertag-site2-target or target-site2-markertag;

(c) isolating the cleaved peptide of (b);

(d) cleaving the isolated peptide of (c) at site2 with the second specific enzyme to produce the target protein; and

(e) isolating the target protein.

The method may include co-expressing a gene encoding the first specific enzyme so that the first specific enzyme cleaves the encoded peptide at site1 in vivo.

It is understood that the first and the second specific enzymes are distinct proteases, each of which may be selected from the list in TABLE I.

In one specific embodiment, the helpertag is a maltose binding protein (MBP), site1 is a peptide sequence recognized by TVMV protease, the markertag is a his₆, and site2 is a peptide sequence recognized by TEV protease.

It is also understood that the steps (c) and (e) may be performed using immobilized metal ion affinity chromatography (IMAC).

In another embodiment, the method of producing a protein includes:

(a) introducing a vector into a cell, wherein the vector comprises a nucleotide sequence encoding a peptide sequence of N-MBP-tvmv-his₆-tev-target, or N-target-tev-his₆-tvmv-MBP, where N is N-terminus of the peptide, MBP is a maltose binding protein, tvmv is a recognition peptide sequence that is cleaved by a TVMV protease, his₆ is a poly-histidine tag, tev is a recognition peptide sequence that is cleaved by a TEV protease, and target is a protein of interest;

(b) co-expressing a gene encoding a TVMV protease;

(c) extracting protein from the cell;

(d) isolating the his₆-tev-target or target-tev-his₆ peptide;

(e) treating the isolated peptide from (d) with the TEV protease; and

(f) isolating the target protein.

The method may be performed using any suitable cells such as bacterial cells, insect cells, animal and plant cells.

Expression of the target proteins as a fusion with MBP results in improved folding and solubility of some target proteins. The in vivo processing of this protein by coexpressed TVMV protease results in production of a his₆-tagged target and a cleaved, untagged MBP. This form of MBP does not bind to the initial IMAC column during purification, and is not carried forward to the second step, thereby eliminating the disruption of the high-throughput purifications. In addition, false positives resulting from an association with MBP are eliminated. In those cases (false positives), the proteins precipitate and will not pass through preliminary, e.g. robotic screens for solubility and reduces wasted effort in the more laborious, large scale purifications.

A recognition sequence may include any protein sequence cleaved specifically by a protease, for example, sequences cleaved by any protease listed in Table 1 or 2, or by similar proteases possessing high selectivity for extended amino acid sequences.

A schematic of an embodiment of the purification methodology is illustrated below.

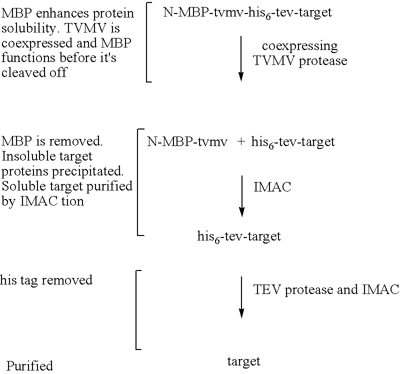

### BRIEF DESCRIPTION OF THE DRAWINGS

FIG. 1 is a schematic illustration of pMCSG vectors, pMCSG7 and derivatives. Vectors are based on the pET system of vectors. Following the T7 promoter, lac operator and ribosome binding site (RBS) of pET-30 Xa/LIC (Novagen, Inc.). (A): pMCSG7 encodes a leader sequence consisting of a his₆-tag, a spacer and the TEV protease recognition sequence followed by a LIC region based on a central SspI site. Restriction sites within and around the expression region sites, BglII and KpnI, allow insertion of modules or replacement sequences into the leader, or transfer of the entire region to different vector backbones. (B): Modifications pMCSG 8, 9, 10, 16, 17, 20 have inserted components: pMCSG8, S-loop (Donnelly et al. (2001)); pMCSG9, MBP; pMCSG10, Glutathione-S-Transferase (GST); pMCSG16, AviTag. For pMCSG17 and pMCSG20 the his₆tag is replaced by S-tag or S-tag-GST, respectively.

FIG. 2 shows improved solubility of proteins produced by pMCSG9 and results of purification of the improved proteins. (A): Effect of insertion of MBP into the leader sequence of 131 proteins on solubility. Proteins that were poorly soluble when expressed in pMCSG7 (having the Solubility Score 1) were produced from pMCSG9 with the leader his₆-MBP-TEV site. Of these, 59 (45%) were improved to Solubility Score 2 or 3, which are sufficiently soluble to proceed to purification. (B): Production of highly soluble target proteins (Solubility Score 3) after partial purification when produced from pMCSG7 (gray bars) or pMCSG9 (black bars). Because the his₆-MBP leader interfered with the second purification step (see main text), yields were calculated after IMAC-I by adjusting the yield of fusion proteins for the portion of their mass due to the leader sequence. (C): Purification of APC25420 produced from pMCSG9 using standardized purification protocols in practice at the MCSG. Lanes are: 1) Molecular weight markers, 2) cell extract (applied to IMAC-I), 3) IMAC-I flow-through, 4) IMAC-I wash, 5) IMAC-I eluate, 6) TEV treated eluate (applied to IMAC-II), 7) IMAC-II flow-through, 8) IMAC-II, wash. After elution of the his₆-MBP-target fusion protein (lane 4) cleavage with TEV protease generates the larger his₆-MBP and smaller target protein (lane 5). Because if its abundance and lower affinity for the IMAC resin, his₆-MBP fails to bind to IMAC-II and elutes with the target protein (lane 8). (D): Two target proteins are illustrated. Lanes 1 and 3 show the mixture of his-tagged MBP (upper band) and target protein that results from TEV cleavage of the fusion protein purified on the first IMAC column. Lanes 2 and 4 show the material eluted from the second IMAC column, which is intended to remove his tagged helper proteins or peptide. The higher molecular weight his-tagged MBP failed to bind to the column, and the final product is highly contaminated with it.

FIG. 3 is a schematic illustration of pMCSG19, a dual tag (MBP and His), dual protease site (TVMV and TEV) vector designed for HTP purifications. Vector pMCSG19 encodes a leader sequence that begins with the untagged MBP protein followed by a TVMV protease site. Beyond this site the leader is identical to that of pMCSG7; after cleavage of proteins expressed from this vector with TVMV, the product is essentially identical to that from pMCSG7 and can be purified by identical protocols.

FIG. 4 shows validation of pMCSG19 for protein expression and in vivo processing. Induction of BL21(DE3) cells containing pMCSG19 alone (lane 1) or with pRK1037, which produced TVMV protease constitutively (lane 2). In the absence of an inserted gene, pMCSG19 produces MBP followed by the TVMV recognition sequence, a his₆-tag, and the TEV recognition sequence, a 45,293 Dalton protein. In the presence of TVMV protease, the C-terminal his-tag, TEV site and 5 additional amino acids encoded by the vector are cleaved, reducing the protein's molecular weight by 2,955 Daltons.

FIG. 5 shows the expression and in vivo processing of 18 proteins in pMCSG19. 18 proteins that failed to give good yields of pure protein when produced from pMCSG9 were produced in pMCSG19 in an auto-inducing medium at 37° C. and analyzed for solubility. All 18 were successfully processed in vivo by TVMV, generating free MBP (the band present in all lanes near the middle of the lane) and all gave a smaller target protein of the expected molecular weight. In some cases, incomplete processing occurred, as indicated by presence of the fusion protein, the additional bands of higher molecular weight than MBP.

FIG. 6 shows a solubility screen of 18 proteins (1, APC22819; 2, APC22808; 3, APC23402; 4, APC23431; 5, APC23256; 6, APC22906; 7, APC23852; 8, APC24034; 9, APC24155; 10, APC24177; 11, APC24238; 12, APC24253; 13, APC25385; 14, APC25420; 15, APC25436; 16, APC25439; 17, APC23650; 18, APC23645. See http://www.mcsg.anl.gov/ for details.) produced in pMCSG19. The 18 proteins introduced into pMCSG19 were produced under screening conditions, in LB at 20° C., and analyzed for solubility. Soluble fractions (upper) contained variable amounts of the target proteins. All proteins were processed in vivo efficiently, as shown by the predominant band of MBP seen in all lanes. Many of the target proteins, however, were more abundant in the insoluble fractions (lower), in some cases sufficiently so to cause precipitation of the fusion protein, seen in the bands of higher molecular weight. MBP was also found in these fractions, presumably arising from cleavage of precipitated fusion proteins by TVMV. Expression from pMCSG19 eliminates possible false positives that expression without removal of MBP causes. That is, in normal expression with pMCSG9—which gives histag-MBP-TEV-target—proteins appear to be more soluble than they really are, and their true nature only is revealed after purification and removal of MBP with TEV protease. With pMCSG19 and the in vivo coexpression of TVMV protease, MBP is removed early, inside the cells, and if the target is truly insoluble, it precipitates. The proteins present in high abundance in the upper panel are much more likely to be purified and crystallized successfully, and can be done so by standard, high-throughput procedures.

FIG. 7 shows the expression of the selenomethionyl form of six proteins (1, APC23431; 2, APC24253; 3, APC25385; 4, APC25420; 5, APC25436; 6, APC25439. See http://www.mcsg.anl.gov/ for details.) in pMCSG19. Production of 6 of the target proteins from pMCSG19 in minimal medium containing selenomethionine at 20° C. resulted in much better solubility. Most of the target was found in the soluble fraction (A) for all but one protein (two experiments were performed for protein 1) and no uncleaved fusion proteins were seen under these conditions. The small amount of MBP in the insoluble fractions is attributed to carry over from the soluble fraction.

FIG. 8 shows the pass-through and eluted fractions from first IMAC column. Robotic processing of four of the proteins shown in FIG. 6 through the first IMAC step showed that the resulting partially purified protein was free of contamination with MBP. In all cases, the lanes are, from left to right; extract applied to the IMAC column, pass through material (in all cases including predominant bands of MBP and the low molecular weight lysozyme used in cell lysis), wash fractions, and the eluted fraction.

FIG. 9 shows the purification fractions of the target protein APC25420 (http://www.mcsg.anl.gov/) produced from pMCSG9 (A) and pMCSG19 (B). Lanes for IMAC1 are: 1, load; 2, pass through; 3, wash; 4, eluate; 5, cleaved with TEV protease. Lanes for IMAC2 are: 6, load; 7, pass through; 8, wash. For the protein produced in pMCSG9, lane one contains the his-tagged-MBP-target fusion protein (protein A) which eluted intact (lane number 4). Cleavage with TEV protease generates his-tagged MBP (protein B) and the untagged target (protein C). During IMAC2, both proteins pass through the column, resulting in contaminated product (lanes 7 and 8). For the protein produced in pMCSG19, lane one contains untagged MBP (protein D) and the his-tagged target (protein E). Untagged MBP passes through the IMAC1 column (lane 2) and his-tagged target elutes without contamination by MBP (lane 4). Cleavage with TEV protease gives untagged target, which passes through IMAC2 to give pure product (lanes 7 and 8).

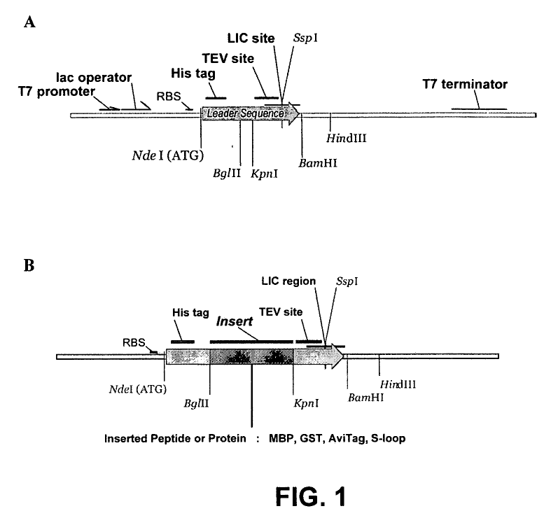

### DETAILED DESCRIPTION

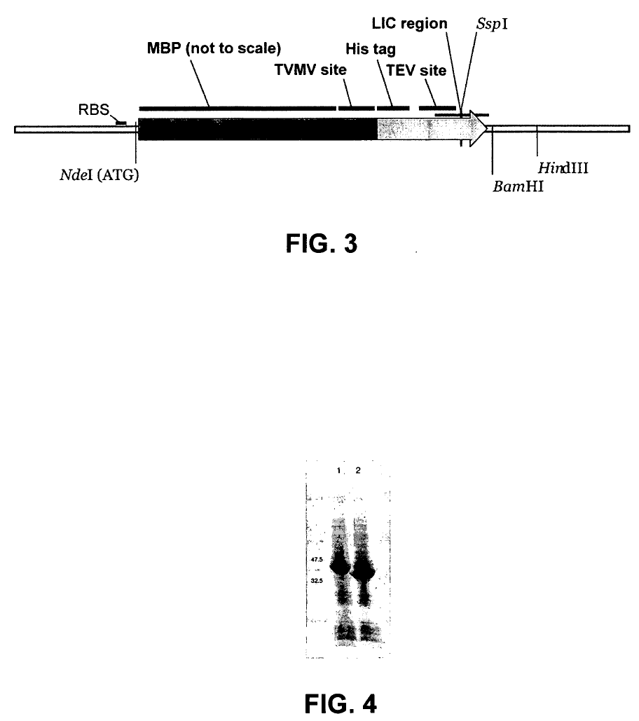

A novel vector, pMCSG19, is designed to allow stepwise removal of the MBP and his-tags and improves protein solubility (FIG. 3). This vector encodes an N-terminal, untagged MBP followed by the recognition sequence for a different, highly specific protease, for example, a specific plant viral protease such as the tobacco vein mottling virus (TVMV) protease, which is then followed by a standard his₆-tag and the TEV protease site. Initial cleavage of the expressed fusion protein with TVMV protease prior to the evaluation of solubility and purification eliminates the false positives that occur with MBP fusions, which are detected and not carried forward to purification. His-tagged MBP is never formed, and standard purification protocols easily separate the his-tagged target protein from untagged MBP in the first IMAC step. After separation, cleavage with TEV and the second IMAC process by standard protocols result in the target protein being free of contamination by MBP. In addition, this process can be streamlined by co-expression of TVMV protease during expression of the target protein, resulting in in vivo cleavage of MBP. The resulting target protein is identical to that expressed from pMCSG7 (except for the presence of an N-terminal serine residue instead of methionine), and is purified without any modification of standard protocols.

The methods and compositions disclosed use the ability of proteases to process polypeptides inside the host cell. The utility of these constructs extends beyond the issues of false solubility and high-throughput purification addressed by the vector designated pMCSG19. Alternative helper components could reduce the toxicity of targets, direct their posttranslational modification, or provide partner proteins required for stability or function of the target protein. A single cloning results in co-expressing the fusion protein and/or the modifying protein. Tags may be designed for applications other than purification, such as detection, transport, or incorporation into combinatorial analyses such as phage display. Any proteases could be used for the in vivo processing, including ones for maturation or activation of the target protein itself by specific proteolysis. For example, controlled cleavage of the polypeptide construct in vivo can activate receptors, enzymes, regulate replication, transcription, translation and other important cellular process.

Construction of pMCSG9. The vector pMCSG9 was constructed by inserting the gene encoding MBP into the KpnI site of vector pMCSG7. The MBP encoding region was generated by PCR using plasmid pRK793 (Kapust, R. B. et al. 2001) as template (a generous gift from David Waugh) and the primers: 5′-TTTTAGATCTGATGTCCCCTATACTAGGTTATTGG (SEQ ID NO: 1) and 5′-TTTTGGTACCTGGGATATCGTAATCATCCGATTTTGGAGGATGGT (SEQ ID NO: 2) (purchased from the Howard Hughes Medical Institute-Keck Laboratory of Yale University, New Haven, Conn.). The vector was digested with KpnI and dephosphorylated with calf intestinal phosphatase (Promega, Corp., Madison, Wis.), and ligated to KpnI-treated PCR product. The resulting plasmids were screened for orientation and expression of a protein of the molecular weight expected for his-tagged-MBP (the product of the vector before introduction of a target gene), and the expression region of a positive candidate was sequenced to verify the identity of MBP with that encoded by pRK793. In addition, during restriction analysis it was found that a portion of the vector near the Ap^{R} gene was slightly larger than anticipated, both in pMCSG9 and pMCSG7. Sequencing of this region revealed that a mutation, most likely selected during construction of pMCSG7, resulting in retention of 129 bases additional bases of the parental vector, pET21a.

Construction and validation of pMCSG19. The sequence encoding MBP followed by the TVMV protease site and the his₆ affinity tag was amplified from the vector pRK1035 by PCR using the primers 5′-TTAAACATATGAAATCGAAGAAGG and 5′-TTATAGGATCCACGCCAGAAGAGTGATGATGATGGTG, and introduced into the NdeI and BlgII sites of pMCSG7 to give pMCSG19. Successful construction of the vector was confirmed by restriction analysis of the vector with PvuI, which cleaves both the parental vector and the MBP gene once. Two fragments of the expected size were observed. Functionality of the vector in expression and in vivo processing was verified by introduction of the vector into BL21 (DE3) cells that contained the plasmid pRK1037 or cells lacking this plasmid. Without insertion of a target protein into the LIC site, pMCSG19 is expected to produce MBP appended with a C-terminal TVMV protease recognition sequence, a his₆-tag, the TEV protease recognition sequence, and 5 amino acids encoded by the unused LIC region (which includes a stop codon). Introduction of each construct resulted in production of a protein of approximately 45,293 Da, the expected size for the modified MBP's, but in the cells which also contained pRK1037 the protein was approximately 2,955 Da smaller as a result of cleavage of the C-terminal TVMV site and loss of the subsequent amino acids. (FIG. 4).

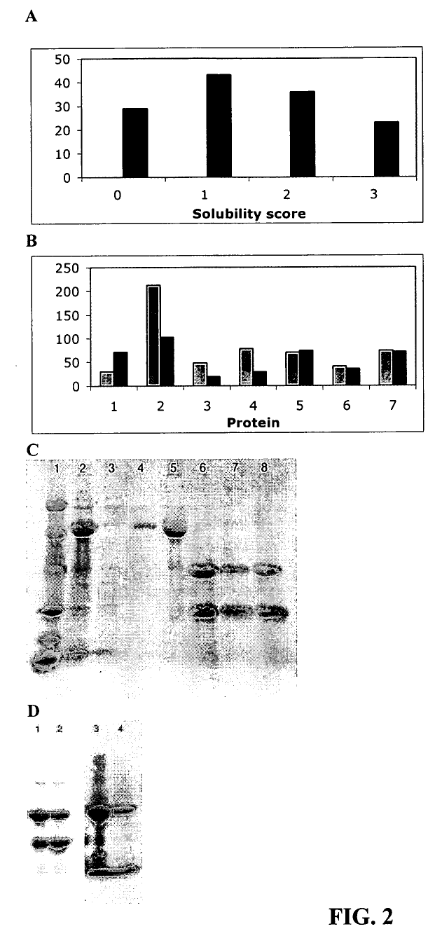

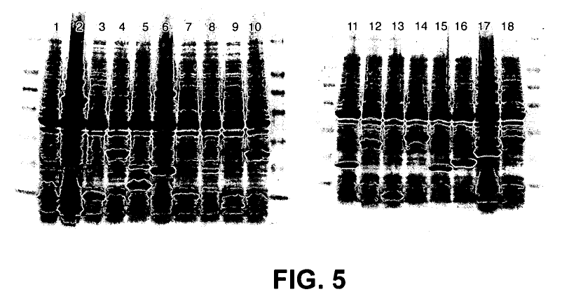

LIC, in vivo processing, and solubility of problematic target proteins. Eighteen target proteins were chosen for evaluation in pMCSG19. These targets had produced poorly soluble proteins when produced from pMCSG7 (his-tag only) but gave soluble products in pMCSG9. However, none generated sufficient, pure material for crystallization after purification by the standard protocols described in the Materials and Methods. Some precipitated after cleavage with TEV to remove the fused his₆-MBP. For those that did not precipitate, purification by the standard purification protocols failed to give pure target protein because the his-tagged MBP failed to bind sufficiently well to the second IMAC column, resulting in contamination of the final product with his₆-MBP (FIGS. 2C and 2D) and necessitating additional purification steps. The PCR products used to introduce these genes into the earlier vectors were introduced into pMCSG19 and transformed into DH5α cells. Plasmid DNA prepared from one colony from each transformation introduced into BL21 (DE3) cells containing the plasmid pRK1037, and a resulting colony was analyzed for expression after overnight growth in autoinducing medium. All eighteen produced a protein of the expected size (FIG. 5). This experiment also showed that the co-produced TVMV protease efficiently processed all the fusion protein in vivo generating MBP (the intermediate molecular weight band present in all lanes) and the target protein, all of which were of lower molecular weight in this experiment. In some cases, a portion of the fusion protein remained uncleaved (see at a higher molecular weight).

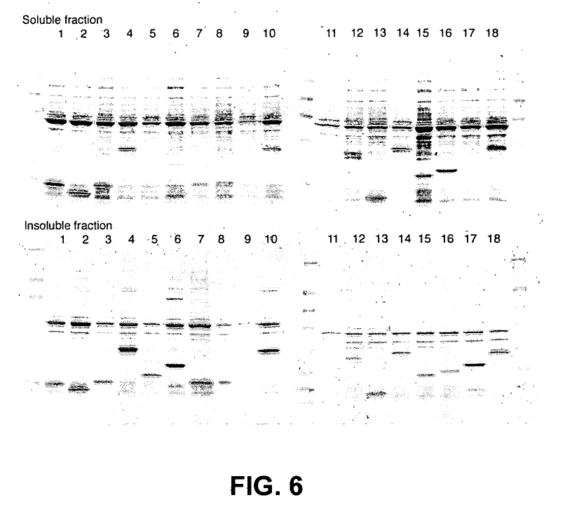

Solubility analysis of targets expressed at 20° C. in LB medium (screening conditions) indicated that many were rendered only partially soluble by their transient association with MBP. (FIG. 6, upper). In several cases, no soluble target was detected. In many cases, the majority of the protein was insoluble (FIG. 6, lower), and in some cases the uncleaved fusion protein was also present in the insoluble fraction.

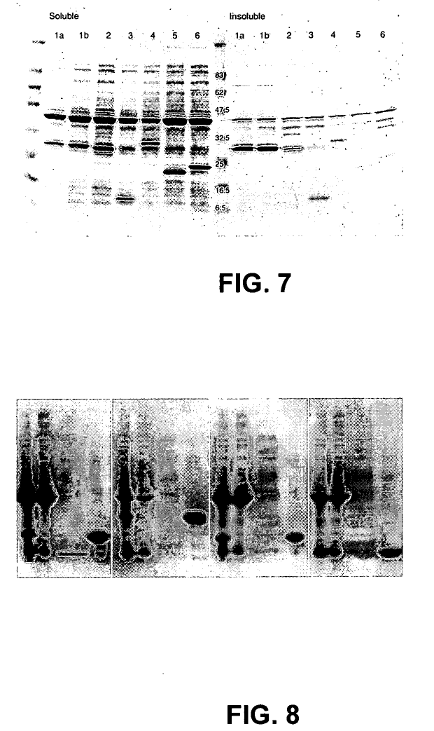

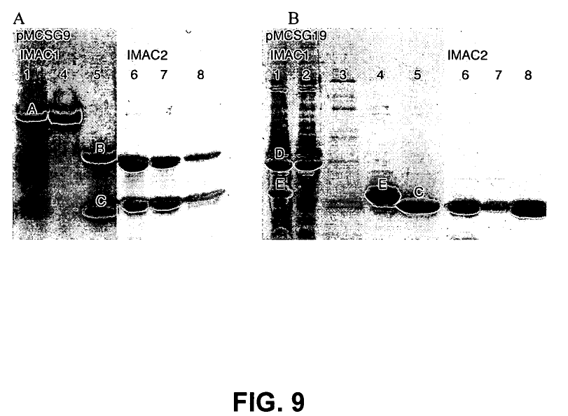

Production and purification of selenomethionyl proteins. Six soluble or partially soluble proteins were expressed as their selenomethionyl form in minimal medium at 20° C. (FIG. 7). Good yields of soluble target protein were obtained for all six. Those proteins that were only moderately soluble under the screening conditions gave much better yields of soluble target in these experiments and a better distribution between soluble and insoluble fractions. In no case was there any evidence for incomplete cleavage by TVMV protease. Purification of four of these proteins by the standard high-throughput protocols resulted in complete removal of the untagged MBP in the first IMAC step (FIG. 8). The value of the dual-tag, dual-protease strategy is illustrated by comparison of fractions from purification of one of the target proteins produced in both vectors (FIG. 9). When the target was expressed from pMCSG9, the his₆ and MBP tagged target is retained by the first IMAC column and eluted intact (FIG. 9A, lane 4). TEV cleavage then generates untagged target and a stoichiometric amount of his-tagged MBP (lane 5), which is bound poorly to the second IMAC column and contaminates the final product (lanes 6-8). In contrast, untagged MBP generated by pMCSG19 passes through the first IMAC column (FIG. 9B, lane 2); the eluted target protein (FIG. 9B, lane 4) is completely free of MBP. Treatment with TEV protease generated untagged target (lane 5), and IMAC2 removes traces of host proteins that bound to the first IMAC column (lanes 6-8), giving target protein of sufficient purity for crystallization.

Constitutive, low level expression of TVMV directed by the plasmid pRK1037 (D. Waugh, purchased from Science Reagents, Inc.) efficiently cleaved the tvmv site following the MBP protein produced by pMCSG19.

The methods and compositions disclosed allow production of two proteins/polypeptides, one tagged, the other not, to enhance any property of either of the two proteins/polypeptides. The specific vector, pMCSG19, is of immediate use by laboratories using the MCSG vectors. The design N-MBP-tvmv-his₆-tev-target can easily be incorporated into different parental vectors, such as pACYCDuet-1 (Novagen, Inc., Madison, Wis.) or Gateway vectors (Invitrogen, Inc., Carlsbad, Calif.) using routine techniques and will be of use to any laboratory involved in producing proteins of limited solubility. The generic designs N-helper-site1-tag-site2-target and N-target-site2-tag-site1-helper will have broad applications in numerous research areas.

Sequence of vector pMCSG19: The coding sequence is on the complementary strand, from bases 1443 through the LIC (ligation independent cloning) SspI site at 219 (where the target gene is inserted by LIC). SEQ ID NO: 3:

ATCCGGATATAGTTCCTCGTTTCAGCAAAAAACCCCTCAAGACCCGTTTAGAGGCCCCAAGG
GGTTATGCTAGTTATTGCTCAGCGGTGGCAGCAGCCAACTCAGCTTCCTTTCGGGCTTTGTTAGCAGC
CGGATCTCAGTGGTGGTGGTGGTGGTGCTCGAGTGCGGCCGCAAGCTTGTCGACGGAGCTGGAATTCg
gatccGTTATGCACTTCCAATATTGGATTGGAAGTACAGGTTCTCggtaccCaGATCCACGCCAGAag
agtgatgatgatggtggtgagaCTGGAAACGCACGGTTTCCGAGCCTGCTTTTTTGTACAAACTTGTG
ATCGAATTAGTCTGCGCGTGTTTCAGGGCTTCATCGACAGTCTGACGACCGCTGGCGGCGTTGATCAC
GGCAGTACGCACGGCATACCAGAAAGCGGACATCTGCGGGATGTTCGGCATGATTTCACCTTTCTGGG
CGTTTTCCATGGTGGCGGCAATACGTGGATCTTTCGCCAACTCTTCCTCGTAAGACTTCAGCGCTACG
GCACCCAGCGGTTTGTCTTTATTAACCGCTTCCAGACCTTCATCAGTCAGCAGATAGTTTTCGAGGAA
CTCTTTTGCCAGCTCTTTGTTCGGACTGGCGGCGTTAATACCTGCGCTCAGCACGCCAACGAACGGTT
TGGATGGTTGACCCTTGAAGGTCGGCAGTACCGTTACACCATAATTCACTTTGCTGGTGTCGATGTTG
GACCATGCCCACGGGCCGTTGATCGTCATCGCTGTTTCGCCTTTATTAAAGGCAGCTTCTGCGATGGA
GTAATCGGTGTCTGCATTCATGTGTTTGTTTTTAATCAGGTCAACCAGGAAGGTCAGACCCGCTTTCG
CGCCAGCGTTATCCACGCCCACGTCTTTAATGTCGTACTTGCCGTTTTCATAGTTGAACGCATAACCC
CCGTCAGCAGCAATCAGCGGCCAGGTGAAGTACGGTTCTTGCAGGTTGAACATCAGCGCGCTCTTACC
TTTCGCTTTCAGTTCTTTATCCAGCGCCGGGATCTCTTCCCAGGTTTTTGGCGGGTTCGGCAGCAGAT
CTTTGTTATAAATCAGCGATAACGCTTCAACAGCGATCGGGTAAGCAATCAGCTTGCGGTTGTAACGT
ACGGCATCCCAGGTAAACGGATACAGCTTGTCCTGGAACGCTTTGTCCGGGGTGATTTCAGCCAACAG
GCCAGATTGAGCGTAGCCAAACCAGCGGTCGTGTGCCCAGAAGATAATGTCAGGGCCATCGCCAGTTG
CCGCAACCTGTGGGAATTTCTCTTCCAGTTTATCCGGATGCTCAACGGTGACTTTAATTCCGGTATCT
TTCTCGAATTTCTTACCGACTTCAGCGAGACCGTTATAGCCTTTATCGCCGTTAATCCAGATTACCAG
TTTACCTTCTTCGATTTTCATatgTATATCTCCTTCTTAAAGTTAAACAAAATTATTTCTAGAGGGGA
ATTGTTATCCGCTCACAATTCCCCTATAGTGAGTCGTATTAATTTCGCGGGATCGAGATCGATCTCGA
TCCTCTACGCCGGACGCATCGTGGCCGGCATCACCGGCGCCACAGGTGCGGTTGCTGGCGCCTATATC
GCCGACATCACCGATGGGGAAGATCGGGCTCGCCACTTCGGGCTCATGAGCGCTTGTTTCGGCGTGGG
TATGGTGGCAGGCCCCGTGGCCGGGGGACTGTTGGGCGCCATCTCCTTGCATGCACCATTCCTTGCGG
CGGCGGTGCTCAACGGCCTCAACCTACTACTGGGCTGCTTCCTAATGCAGGAGTCGCATAAGGGAGAG
CGTCGAGATCCCGGACACCATCGAATGGCGCAAAACCTTTCGCGGTATGGCATGATAGCGCCCGGAAG
AGAGTCAATTCAGGGTGGTGAATGTGAAACCAGTAACGTTATACGATGTCGCAGAGTATGCCGGTGTC
TCTTATCAGACCGTTTCCCGCGTGGTGAACCAGGCCAGCCACGTTTCTGCGAAAACGCGGGAAAAAGT
GGAAGCGGCGATGGCGGAGCTGAATTACATTCCCAACCGCGTGGCACAACAACTGGCGGGCAAACAGT
CGTTGCTGATTGGCGTTGCCACCTCCAGTCTGGCCCTGCACGCGCCGTCGCAAATTGTCGCGGCGATT
AAATCTCGCGCCGATCAACTGGGTGCCAGCGTGGTGGTGTCGATGGTAGAACGAAGCGGCGTCGAAGC
CTGTAAAGCGGCGGTGCACAATCTTCTCGCGCAACGCGTCAGTGGGCTGATCATTAACTATCCGCTGG
ATGACCAGGATGCCATTGCTGTGGAAGCTGCCTGCACTAATGTTCCGGCGTTATTTCTTGATGTCTCT
GACCAGACACCCATCAACAGTATTATTTTCTCCCATGAAGACGGTACGCGACTGGGCGTGGAGCATCT
GGTCGCATTGGGTCACCAGCAAATCGCGCTGTTAGCGGGCCCATTAAGTTCTGTCTCGGCGCGTCTGC
GTCTGGCTGGCTGGCATAAATATCTCACTCGCAATCAAATTCAGCCGATAGCGGAACGGGAAGGCGAC
TGGAGTGCCATGTCCGGTTTTCAACAAACCATGCAAATGCTGAATGAGGGCATCGTTCCCACTGCGAT
GCTGGTTCCCAACGATCAGATGGCGCTGGGCGCAATGCGCGCCATTACCGAGTCCGGGCTGCGCGTTG
GTGCGGATATCTCGGTAGTGGGATACGACGATACCGAAGACAGCTCATGTTATATCCCGCCGTTAACC
ACCATCAAACAGGATTTTCGCCTGCTGGGGGAAACCAGCGTGGACCGCTTGGTGCAACTCTCTCAGGG
CCAGGCGGTGAAGGCCAATCAGCTGTTGCCCGTCTCACTGGTGAAAAGAAAAACCACCCTGGCGCCCA
ATACGCAAACCGCCTCTCCCCGCGCGTTGGCCGATTCATTAATGCAGCTGGCACGACAGGTTTCCCGA
CTGGAAAGCGGGCAGTGAGCGGAACGGAATTAATGTAAGTTAGCTCACTGATTAGGCACCGGGATCTC
GACCGATGCCCTTGAGAGCCTTCAACCCAGTCAGCTCCTTCCGGTGGGCGCGGGGCATGACTATCGTC
GCCGCACTTATGACTGTCTTCTTTATCATGCAACTCGTAGGACAGGTGCCGGCAGCGCTCTGGGTCAT
TTTCGGCGAGGACCGCTTTCGCTGGAGCGCGACGATGATCGGCCTGTCGCTTGCGGTATTCGGAATCT
TGCACGCCCTCGCTCAAGCCTTCGTCACTGGTCGCGCCACCAAACGTTTCGGCGAGAAGCAGGCCATT
ATCGCCGGCATGGCGGCCCCACGGGTGCGCATGATCGTGCTCCTGTGGTTGAGGACCCGGCTAGGCTG
GCGGGGTTGCCTTACTGGTTAGCAGAATGAATCACCGATACGCGAGCGAACGTGAAGCGACTGCTGCT
GGAAAACGTCTGCGACCTGAGCAACAACATGAATGGTCTTCGGTTTCCGTGTTTCGTAAAGTCTGGAA
ACGCGGAAGTCAGCGGCCTGCACCATTATGTTCCGGATCTGCATCGCAGGATGCTGCTGGCTACCCTG
TGGAACACCTACATCTGTATTAACGAAGCGCTGGCATTGACCCTGAGTGATTTTTCTCTGGTCCCGCC
GCATCCATACCGCCAGTTGTTTACCCTCACAACGTTCCAGTAACCGGGCATGTTCATCATCAGTAACC
CGTATCGTGAGCATCCTCTCTCGTTTCATCGGTATCATTACCCCCATGAACAGAAATCCCCCTTAGAC
GGAGGCATCAGTGACCAAACAGGAAAAAACCGCCCTTAACATGGCCCGCTTTATCAGAAGCCAGACAT
TAACGCTTCTGGAGAAACTCAACGAGCTGGACGCGGATGAACAGGGAGACATCTGTGAATCGCTTCAC
GACCACGCTGATGAGCTTTACCGCAGCTGCCTCGCGCGTTTCGGTGATGACGGTGAAAACCTCTGACA
CATGCAGCTCCCGGAGACGGTCACAGCTTGTCTGTAAGCGGATGCCGGGAGCAGACAAGCCCGTCAGG
GCGCGTCAGCGGGTGTTGGCGGGTGTCGGGGCGCAGCCATGACCCAGTCACGTAGCGATAGCGGAGTG
TATACTGGCTTAACTATGCGGCATCAGAGCAGATTGTACTGAGAGTGCACCATATATGCGGTGTGAAA
TAGCGCACAGATGCGTAAGGAGAAAATACCGCATCAGGCGCTCTTCCGCTTCCTCGCTCACTGACTCG
CTGCGCTCGGTCGTTCGGCTGCGGCGAGCGGTATCAGCTCACTCAAAGGCGGTAATACGGTTATCCAC
AGAATCAGGGGATAACGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAAAAATCGACGCTCA
AGTCAGAGGTGGCGAAACCCGACAGGACTATAAAGATACCAGGCGTTTCCCCCTGGAAGCTCCCTCGT
GCGCTCTCCTGTTCCGACCCTGCCGCTTACCGGATACCTGTCCGCCTTTCTCCCTTCGGGAAGCGTGG
CGCTTTCTCATAGCTCACGCTGTAGGTATCTCAGTTCGGTGTAGGTCGTTCGCTCCAAGCTGGGCTGT
GTGCACGAACCCCCCGTTCAGCCCGACCGCTGCGCCTTATCCGGTAACTATCGTCTTGAGTCCAACGC
GGTAAGACACGACTTATCGCCACTGGCAGCAGCCACTGGTAACAGGATTAGCAGAGCGAGGTATGTAG
GCGGTGCTACAGAGTTCTTGAAGTGGTGGCCTAACTACGGCTACACTAGAAGGACAGTATTTGGTATC
TGCGCTCTGCTGAAGCCAGTTACCTTCGGAAAAAGAGTTGGTAGCTCTTGATCCGGCAAACAAACCAC
CGCTGGTAGCGGTGGTTTTTTTGTTTGCAAGCAGCAGATTACGCGCAGAAAAAAAGGATCTCAAGAAG
ATCCTTTGATCTTTTCTACGGGGTCTGACGCTCAGTGGAACGAAAACTCACGTTAAGGGATTTTGGTC
ATGAGATTATCAAAAAGGATCTTCACCTAGATCCTTTTAAATTAAAAATGAAGTTTTAAATCAATCTA
AAGTATATATGAGTAAACTTGGTCTGACAGTTACCAATGCTTAATCAGTGAGGCACCTATCTCAGCGA
TCTGTCTATTTCGTTCATCCATAGTTGCCTGACTCCCCGTCGTGTAGATAACTACGATACGGGAGGGC
TTACCATCTGGCCCCAGTGCTGCAATGATACCGCGAGACCCACGCTCACCGGCTCCAGATTTATCAGC
AATAAACCAGCCAGCCGGAAGGGCCGAGCGCAGAAGTGGTGCTGCAACTTTATCCGCCTCCATCCAGT
CTATTAATTGTTGCCGGGAAGCTAGAGTAAGTAGTTCGCCAGTTAATAGTTTGCGCAACGTTGTTGCC
ATTGCTGCAGGCATCGTGGTGTCACGCTCGTCGTTTGGTATGGCTTCATTCAGCTCCGGTTCCCAACG
ATCAAGGCGAGTTACATGATCCCCCATGTTGTGCAAAAAAGCGGTTAGCTCCTTCGGTCCTCCGATCG
TTGTCAGAAGTAAGTTGGCCGCAGTGTTATCACTCATGGTTATGGCAGCACTGCATAATTCTCTTACT
GTCATGCCATCCGTAAGATGCTTTTCTGTGACTGGTGAGTACTCAACCAAGTCATTCTGAGAATAGTG
TATGCGGCGACCGAGTTGCTCTTGCCCGGCGTCAATACGGGATAATACCGCGCCACATAGCAGAACTT
TAAAAGTGCTCATCATTGGAAAACGTTCTTCGGGGCGAAAACTCTCAAGGATCTTACCGCTGTTGAGA
TCCAGTTCGATGTAACCCACTCGTGCACCCAACTGATCTTCAGCATCTTTTACTTTCACCAGCGTTTC
TGGGTGAGCAAAAACAGGAAGGCAAAATGCCGCAAAAAAGGGAATAAGGGCGACACGGAAATGTTGAA
TACTCATACTCTTCCTTTTTCAAattattgaagcatttatcagggttattgtctcatgagcggataca
tatttgaatgtatttagaaaaataaacaaataggggttccgcgcacatttccccgaaaagtgccacct
aaattgtaagcgttaatGTGAACCATCACCCTAATCAAGTTTTTTGGGGTCGAGGTGCCGTAAAGCAC
TAAATCGGAACCCTAAAGGGAGCCCCCGATTTAGAGCTTGACGGGGAAAGCCGGCGAACGTGGCGAGA
AAGGAAGGGAAGAAAGCGAAAGGAGGGGGCGCTAGGGCGCTGGCAAGTGTAGCGGTCACGCTGCGCGT
AACCACCACACGCGCCGCGGTTAATGCGCGGCTACAGGGCGCGTCCCATTCGCCA

Coding Region of pMCSG19:

Sequence of complementary strand of pMCSG19 encoding the leader sequence consisting of MBP-tvmv-site-his6-tag-tev site followed by the LIC SspI site (first three bases AAT shown). Genes introduced by LIC begin after this sequence. SEQ ID NO 4:

ATGAAAATCGAAGAAGGTAAACTGGTAATCTGGATTAACGGCGATAAAGGCTATAACGGTCT
CGCTGAAGTCGGTAAGAAATTCGAGAAAGATACCGGAATTAAAGTCACCGTTGAGCATCCGGATAAAC
TGGAAGAGAAATTCCCACAGGTTGCGGCAACTGGCGATGGCCCTGACATTATCTTCTGGGCACACGAC
CGCTTTGGTGGCTACGCTCAATCTGGCCTGTTGGCTGAAATCACCCCGGACAAAGCGTTCCAGGACAA
GCTGTATCCGTTTACCTGGGATGCCGTACGTTACAACGGCAAGCTGATTGCTTACCCGATCGCTGTTG
AAGCGTTATCGCTGATTTATAACAAAGATCTGCTGCCGAACCCGCCAAAAACCTGGGAAGAGATCCCG
GCGCTGGATAAAGAACTGAAAGCGAAAGGTAAGAGCGCGCTGATGTTCAACCTGCAAGAACCGTACTT
CACCTGGCCGCTGATTGCTGCTGACGGGGGTTATGCGTTCAAGTATCAAAACGGCAAGTACGACATTA
AAGACGTGGGCGTGGATAACGCTGGCGCGAAAGCGGGTCTGACCTTCCTGGTTGACCTGATTAAAAAC
AAACACATGAATGCAGACACCGATTACTCCATCGCAGAAGCTGCCTTTAATAAAGGCGAAACAGCGAT
GACCATCAACGGCCCGTGGGCATGGTCCAACATCGACACCAGCAAAGTGAATTATGGTGTAACGGTAC
TGCCGACCTTCAAGGGTCAACCATCCAAACCGTTCGTTGGCGTGCTGAGCGCAGGTATTAACGCCGCC
AGTCCGAACAAAGAGCTGGCAAAAGAGTTCCTCGAAAACTATCTGCTGACTGATGAAGGTCTGGAAGC
GGTTAATAAAGACAAACCGCTGGGTGCCGTAGCGCTGAAGTCTTACGAGGAAGAGTTGGCGAAAGATC
CACGTATTGCCGCCACCATGGAAAACGCCCAGAAAGGTGAAATCATGCCGAACATCCCGCAGATGTCC
GCTTTCTGGTATGCCGTGCGTACTGCGGTGATCAACGCCGCCAGCGGTCGTCAGACTGTCGATGAAGC
CCTGAAAGACGCGCAGACTAATTCGATCACAAGTTTGTACAAAAAAGCAGGCTCGGAAACCGTGCGTT
TCCAGtctcaccaccatcatcatcactctTCTGGCGTGGATCtGggtaccGAGAACCTGTACTTCCAA
TCCAAT

Materials and Methods.

Construction of pMCSG19. The vector pMCSG19 was constructed by inserting a DNA fragment encoding an untagged MBP followed by the recognition sequence for TVMV protease and a his₆ sequence into the vector pMCSG7 (Stols, et al., 2002). This DNA fragment was generated by PCR using the plasmid pRK1035 as template. (http://mcl1.ncifcrf.gov/waugh_prk1035.html), which was purchased from Scientific Reagents, Inc. This vector contains a region encoding the MBP-TVMV-his₆ sequence. The primers used for the reaction were:

| | |
|---|---|
| TTAAACATATGAAATCGAAGAAGG | (SEQ ID NO:5) |
| and | |
| TTATAGGATCCACGCCAGAAGAGTGATGATGATGGTG. | (SEQ ID NO:6) |

PCR conditions were: denaturatation, 95° C., 1 min; annealing, 46° C., 1 min, and elongation, 68° C., 1.1 min using the enzyme Platinum Pfx polymerase (Invitrogen) in 2× strength reaction buffer with 1 mM Mg++ for 25 cycles. The PCR product was purified by agarose gel electrophoresis and extraction with a QiaEx I kit (Qiagen, Inc., Valencia, Calif.), cleaved with the restriction enzymes NdeI and BamHI then ligated into pMCSG7 which had been treated with NdeI and BglII followed by calf intestinal phosphatase and gel purification as described above. The resulting ligation product was transformed into DH5α cells and plasmids were purified from colonies that grew on LB/ampicillin plates. Insertion and orientation of the PCR fragment was confirmed by restriction analysis with SspI and PvuI.

Cloning genes into pMCSG19. The vector was prepared for LIC using standard protocols (Diekmon, et al., 2002) consisting of cleavage with SspI endonuclease, purification by agarose gel electrophoresis, and treatment with T4 DNA polymerase in the presence of dGTP. Fifteen μg of vector DNA, purified with a Qiagen Plasmid Midi kit (Qiagen, Inc. Valencia, Calif.), was incubated with 75 units of high concentration SspI (New England Biolabs) at 37° C. for 2 h in a reaction volume of 60 μl, then purified following agarose gel electrophoresis using a QiaEx II gel extraction kit. The material was then treated with 40 units of LIC-qualified T4 DNA polymerase (Novagen, Inc., Madison, Wis.) and 4 mM dGTP in a reaction volume of 600 μl. Genes were amplified by PCR with primers encoding the LIC overhang: sense: TACTTCCAATCCAATGCX (SEQ ID NO: 7) followed by the genes' N-terminal sequences, antisense: TTATCCACTTCCAATG (SEQ ID NO: 8) followed by the complement of a stop codon and the C-terminus of the gene, purified with a QIAQuick PCR purification kit (Qiagen, Inc.). Eighteen PCR products encoding proteins which had failed to generate satisfactory amounts of material using standard, high-throughput protocols, were treated with T4 DNA polymerase in the presence of dCTP, annealed to the treated pMCSG19. Following annealing of 100 ng of this material with 50 ng LIC-prepared vector, the resulting plasmids were transformed into DH5α cells Plasmid DNA was isolated from a representative of each transformation.

Expression and in vivo processing of the fusion proteins. To allow in vivo processing of the fusion proteins (Kapust, et al., 1999), the expression plasmids described above were transformed into BL21 (DE3) cells that contained the plasmid pRK1037 (http://mcl1.ncifcrf.gov/waugh_prk1037.html; Scientific Reagents, Inc.). This plasmid carries a gene encoding TVMV protease under control of the 1P_{L}/tetO promoter, which is active in cells such as BL21 (DE3) that do not produce the Tet repressor, resulting in constitutive expression of low levels of TVMV protease (http://mcl1.ncifcrf.gov/waugh_prk1037.html). Transformants were isolated on LB plates containing 100 μg/ml kanamycin (required to maintain pRK1037). For analysis of total protein expression, a colony was transferred into autoinducing medium (Terrific Broth containing a glucose, lactose and glycerol as carbon sources at 0.5, 2 and 5 g/L, respectively (F. W. Studier, personal communication)), grown overnight at 37° C., then lysed according to established protocols (Millard, et al., 2003).

Expression and analysis of solubility. For analysis of soluble protein, a colony was grown at 37° in LB containing ampicillin and kanamycin, as above, to an OD₆₀₀ of 0.5 to 1 when protein synthesis was induced by addition of 1 mM IPTG. After 3 h, cells were harvested and lysed to allow separation of the soluble and insoluble proteins. Briefly, cells were suspended in lysis buffer (50 mM HEPES, pH 7.8, containing 500 mM NaCl, 10 mM imidazole, 10 mM-mercaptoethanol, and 5% glycerol), incubated with recombinant lysozyme and Benzonase (a source of DNase activity) for 30 min at 37°, frozen briefly, then sonicated to lyse the cells. Following centrifugation at 6000×g for 15 min, the supernatant and pellet were separated and analyzed for protein by denaturing gel electrophoresis.

Production and purification of selenomethionyl proteins. The Selenomethionyl proteins were produced in BL21 (DE3), a strain not auxotrophic for methionine, using feedback inhibition of methionine biosynthesis. The selenomethionyl forms of 6 proteins were produced with in vivo processing using published protocols (Stols et al., 2004) as follows. Cultures from glycerol stocks were initiated in LB containing ampicillin and kanamycin as described herein and subcultured after 6 h into 25 ml of M9 medium plus antibiotics in 125 ml baffled erlenmeyer flasks, and grown overnight at 37° with shaking at 300 rpm. The following day the cultures were diluted into 1 L of the same medium and grown at 37° to an OD₆₀₀ of 0.5. At that point, IPTG (1 mM), selenomethionine (60 μg/ml), and a cocktail of 6 amino acids that inhibit the biosynthetic pathways to methionine (leucine, isoleucine, lysine, valine, phenylalanine and threonine, all at 100 μg/ml) were added and the culture was shifted to 20° and incubated overnight. Cells were harvested by centrifugation, suspended in lysis buffer, and processed by established high-throughput protocols to purify target proteins.

Alternatively, cultures were grown in 2-liter polyethylene terephthalate beverage bottles ((Millard, C. S. et al. 2003) containing one liter of non-sterile M9 salts supplemented with glucose, glycerol, amino acids, trace metals and vitamins to increase the cell yield. Amendments were, per liter: glycerol, 5 g; glucose, 4.4 g; non-inhibitory amino acids (L-glutamate, L-aspartate, L-arginine, L-histidine, L-alanine, L-proline, L-glycine, L-serine, L-glutamine, L-asparagine, and L-tryptophan), 200 mg each; trace metal mixture (EDTA, 5 mg; MgCl.6H₂O, 430 mg; MnSO₄.H₂O, 5 mg; NaCl, 10 mg; FeSO₄.7H₂O, 1 mg; Co(NO₃)₂.6H₂O, 1 mg; CaCl₂, 11 mg; ZnSO₄.7H₂O, 1 mg; CuSO₄.5H₂O, 0.1 mg; AlK(SO₄)₂, 0.1 mg; H₃BO₃, 0.1 mg; Na₂MoO₄.2H₂O, 0.1 mg; Na₂SeO₃, 0.01 mg; Na₂WO₄.2H₂O, 0.1 mg; NiCl₂.6H₂O, 0.1 mg); ampicillin, 50 mg; kanamycin, 30 mg; thiamine 1 mg; and vitamin B12, 2.7 mg. Media components other than glycerol were supplied as aliquots of mixed solids in foil packets or as concentrated stock solutions by Medicillin, Inc., Chicago, Ill. (catalog numbers MD045004A, MD045004B, MD045004C, and MD045004E). Cultures were grown at 37° C. to an OD₆₀₀=1-2, when inhibitory amino acids (25 mg each of L-valine, L-isoleucine , L-leucine, L-lysine, L-threonine, L-phenolalanine, and 15 mg of selenomethionine; Medicillin, Inc. catalog number MD045004D) and 1 mM isopropylthio-beta-D-galactoside (IPTG) were added, and the temperature dropped to 20° C. Cultures were incubated overnight, then harvested. by centrifugation. With the supplements, the yield of cells per liter of medium was more than doubled compared to unamended M9 medium reported by Millard, C. S. et al. (2003). Amino acid analysis of four purified proteins resulted in no detectable methionine, indicating greater than 90% incorporation of selenomethionine within the detection limits of the analysis.

This protein extraction and purification process consists of cell lysis by sonication, followed by centrifugation, and semi-robotic purification of his-tagged proteins by three chromatographic steps. The first step, IMAC1, binds and elutes his-tagged target proteins by robotic immobilized metal-ion affinity chromatography (IMAC), followed by the second automated step, desalting by gel filtration. The his-tag is then cleaved from the target protein by incubation with TEV protease modified to have a non-cleavable his-tag cleavage of the his-tag with TEV protease, and secondary subtractive IMAC to remove TEV protease and trace host proteins.

TABLE I
List of viral proteases including GenBank identification numbers
along with their E-value scores that produce significant sequence alignments.

| | | |
|---|---|---|
| gi\|9790345\|ref\|NP_062908.1\| polyprotein [Tobacco etch virus] >gi . . . | 417 | e−115 |
| gi\|23476451\|gb\|AAN27999.1\| polyprotein [Bean common mosaic necro . . . | 414 | e−115 |
| gi\|30961865\|gb\|AAP38183.1\| polyprotein [Bean common mosaic necro . . . | 414 | e−115 |
| gi\|21553929\|ref\|NP_660175.1\| polyprotein [Bean common mosaic nec . . . | 414 | e−115 |
| gi\|609608\|gb\|AAA98577.1\| polyprotein | 414 | e−115 |
| gi\|18621100\|emb\|CAC86160.1\| polyprotein [Bean common mosaic viru . . . | 411 | e−114 |
| gi\|19070509\|gb\|AAL83896.1\| polyprotein [Cowpea aphid-borne mosai . . . | 410 | e−113 |
| gi\|31321957\|gb\|AAM60816.1\| polyprotein precursor [Bean common mo . . . | 410 | e−113 |
| gi\|15055164\|gb\|AAK82883.1\| polyprotein [Turnip mosaic virus] | 410 | e−113 |
| gi\|49387220\|dbj\|BAD24945.1\| polyprotein [Turnip mosaic virus] | 410 | e−113 |
| gi\|33146237\|dbj\|BAC79402.1\| polyprotein [Turnip mosaic virus] | 410 | e−113 |
| gi\|33146245\|dbj\|BAC79406.1\| polyprotein [Turnip mosaic virus] | 410 | e−113 |
| gi\|33146251\|dbj\|BAC79409.1\| polyprotein [Turnip mosaic virus] | 410 | e−113 |
| gi\|33146273\|dbj\|BAC79420.1\| polyprotein [Turnip mosaic virus] | 410 | e−113 |
| gi\|9789712\|ref\|NP_062866.1\| polyprotein [Turnip mosaic virus] >g . . . | 410 | e−113 |
| gi\|15055166\|gb\|AAK82884.1\| polyprotein [Turnip mosaic virus] | 409 | e−113 |
| gi\|49387223\|dbj\|BAD24946.1\| polyprotein [Turnip mosaic virus] | 409 | e−113 |
| gi\|33146275\|dbj\|BAC79421.1\| polyprotein [Turnip mosaic virus] | 409 | e−113 |
| gi\|33146259\|dbj\|BAC79413.1\| polyprotein [Turnip mosaic virus] | 409 | e−113 |
| gi\|33146271\|dbj\|BAC79419.1\| polyprotein [Turnip mosaic virus] | 409 | e−113 |
| gi\|33146247\|dbj\|BAC79407.1\| polyprotein [Turnip mosaic virus] | 409 | e−113 |
| gi\|33146261\|dbj\|BAC79414.1\| polyprotein [Turnip mosaic virus] | 409 | e−113 |
| gi\|22532506\|gb\|AAM09074.1\| polyprotein [Turnip mosaic virus] | 409 | e−113 |
| gi\|33504662\|gb\|AAP48793.1\| polyprotein [Turnip mosaic virus] | 409 | e−113 |
| gi\|1016235\|gb\|AAB53147.1\| polyprotein | 408 | e−113 |
| gi\|1854440\|dbj\|BAA11836.1\| polyprotein [Turnip mosaic virus] >gi . . . | 408 | e−113 |
| gi\|1335724\|gb\|AAB01025.1\| polyprotein | 408 | e−113 |
| gi\|46318075\|gb\|AAS87605.1\| polyprotein [Blackeye cowpea mosaic v . . . | 408 | e−113 |
| gi\|51949946\|ref\|YP_077181.1\| polyprotein [Watermelon mosaic viru . . . | 408 | e−113 |
| gi\|33146277\|dbj\|BAC79422.1\| polyprotein [Turnip mosaic virus] | 408 | e−113 |
| gi\|33146267\|dbj\|BAC79417.1\| polyprotein [Turnip mosaic virus] | 407 | e−113 |
| gi\|33146269\|dbj\|BAC79418.1\| polyprotein [Turnip mosaic virus] | 407 | e−113 |
| gi\|33146235\|dbj\|BAC79401.1\| polyprotein [Turnip mosaic virus] | 407 | e−112 |
| gi\|33146249\|dbj\|BAC79408.1\| polyprotein [Turnip mosaic virus] | 407 | e−112 |
| gi\|33146227\|dbj\|BAC79397.1\| polyprotein [Turnip mosaic virus] | 407 | e−112 |
| gi\|33146253\|dbj\|BAC79410.1\| polyprotein [Turnip mosaic virus] | 407 | e−112 |
| gi\|33146255\|dbj\|BAC79411.1\| polyprotein [Turnip mosaic virus] | 407 | e−112 |
| gi\|33146263\|dbj\|BAC79415.1\| polyprotein [Turnip mosaic virus] | 407 | e−112 |
| gi\|33146265\|dbj\|BAC79416.1\| polyprotein [Turnip mosaic virus] | 407 | e−112 |
| gi\|33146279\|dbj\|BAC79423.1\| polyprotein [Turnip mosaic virus] | 407 | e−112 |
| gi\|33146281\|dbj\|BAC79424.1\| polyprotein [Turnip mosaic virus] | 407 | e−112 |
| gi\|33146229\|dbj\|BAC79398.1\| polyprotein [Turnip mosaic virus] | 407 | e−112 |
| gi\|33146233\|dbj\|BAC79400.1\| polyprotein [Turnip mosaic virus] | 407 | e−112 |
| gi\|33146257\|dbj\|BAC79412.1\| polyprotein [Turnip mosaic virus] | 407 | e−112 |
| gi\|5705963\|gb\|AAB22819.2\| polyprotein [Soybean mosaic virus] >gi . . . | 407 | e−112 |
| gi\|18621102\|emb\|CAC86161.1\| unnamed protein product [Bean common . . . | 407 | e−112 |
| gi\|33146241\|dbj\|BAC79404.1\| polyprotein [Turnip mosaic virus] | 406 | e−112 |
| gi\|33146243\|dbj\|BAC79405.1\| polyprotein [Turnip mosaic virus] | 406 | e−112 |
| gi\|27877111\|dbj\|BAC55871.1\| polyprotein [Soybean mosaic virus] | 405 | e−112 |
| gi\|27877113\|dbj\|BAC55872.1\| polyprotein [Soybean mosaic virus] | 405 | e−112 |
| gi\|12018226\|ref\|NP_072165.1\| polyprotein precursor [Soybean mosa . . . | 405 | e−112 |
| gi\|29372736\|emb\|CAC84443.1\| polyprotein [Soybean mosaic virus] | 405 | e−112 |
| gi\|32452359\|emb\|CAC86162.1\| unnamed protein product [Soybean mos . . . | 405 | e−112 |
| gi\|37528761\|gb\|AAP45048.1\| polyprotein precursor [Soybean mosaic . . . | 405 | e−112 |
| gi\|32265056\|gb\|AAO32625.1\| polyprotein [Soybean mosaic virus] | 405 | e−112 |
| gi\|419498\|pir∥JQ1895 genome polyprotein - turnip mosaic virus >. . . | 405 | e−112 |
| gi\|34304613\|gb\|AAQ63412.1\| polyprotein precursor [Soybean mosaic . . . | 405 | e−112 |
| gi\|33146239\|dbj\|BAC79403.1\| polyprotein [Turnip mosaic virus] | 405 | e−112 |
| gi\|34304611\|gb\|AAQ63411.1\| polyprotein precursor [Soybean mosaic . . . | 404 | e−112 |
| gi\|33146231\|dbj\|BAC79399.1\| polyprotein [Turnip mosaic virus] | 404 | e−112 |
| gi\|222659\|dbj\|BAA01452.1\| polyprotein precursor [Turnip mosaic v . . . | 404 | e−111 |
| gi\|41393050\|emb\|CAD45439.2\| polyprotein [Soybean mosaic virus] | 404 | e−111 |
| gi\|33146223\|dbj\|BAC79395.1\| polyprotein [Turnip mosaic virus] | 404 | e−111 |
| gi\|33146225\|dbj\|BAC79396.1\| polyprotein [Turnip mosaic virus] | 404 | e−111 |
| gi\|281428\|pir∥JQ1662 genome polyprotein - soybean mosaic virus . . . | 403 | e−111 |
| gi\|7682686\|gb\|AAF67344.1\| polyprotein [Soybean mosaic virus] | 403 | e−111 |
| gi\|33146221\|dbj\|BAC79394.1\| polyprotein [Turnip mosaic virus] | 403 | e−111 |
| gi\|9629731\|ref\|NP_045216.1\| polyprotein [Sweet potato feathery m . . . | 402 | e−111 |
| gi\|1304228\|dbj\|BAA07546.1\| polyprotein [Sweet potato feathery mo . . . | 402 | e−111 |
| gi\|51556070\|dbj\|BAD38778.1\| polyprotein [Turnip mosaic virus] | 402 | e−111 |
| gi\|47563873\|dbj\|BAD20396.1\| polyprotein [Turnip mosaic virus] | 402 | e−111 |
| gi\|40311069\|emb\|CAF03595.1\| polyprotein [Soybean mosaic virus] | 402 | e−111 |
| gi\|51556028\|dbj\|BAD38757.1\| polyprotein [Turnip mosaic virus] | 402 | e−111 |
| gi\|40252371\|emb\|CAF02291.1\| polyprotein [Plum pox virus] | 401 | e−111 |
| gi\|47563883\|dbj\|BAD20401.1\| polyprotein [Turnip mosaic virus] | 401 | e−111 |
| gi\|33146219\|dbj\|BAC79393.1\| polyprotein [Turnip mosaic virus] | 401 | e−111 |
| gi\|61336\|emb\|CAA39698.1\| genome polyprotein [Plum pox virus] >gi . . . | 401 | e−111 |
| gi\|47563829\|dbj\|BAD20374.1\| polyprotein [Turnip mosaic virus] | 400 | e−110 |
| gi\|47563887\|dbj\|BAD20403.1\| polyprotein [Turnip mosaic virus] | 400 | e−110 |
| gi\|47563881\|dbj\|BAD20400.1\| polyprotein [Turnip mosaic virus] >g . . . | 400 | e−110 |
| gi\|20153340\|ref\|NP_619667.1\| polyprotein [Lettuce mosaic virus] . . . | 400 | e−110 |
| gi\|47563795\|dbj\|BAD20357.1\| polyprotein [Turnip mosaic virus] | 400 | e−110 |
| gi\|47563875\|dbj\|BAD20397.1\| polyprotein [Turnip mosaic virus] | 400 | e−110 |
| gi\|47563807\|dbj\|BAD20363.1\| polyprotein [Turnip mosaic virus] | 400 | e−110 |
| gi\|47563849\|dbj\|BAD20384.1\| polyprotein [Turnip mosaic virus] | 400 | e−110 |
| gi\|47563791\|dbj\|BAD20355.1\| polyprotein [Turnip mosaic virus] | 400 | e−110 |
| gi\|47563805\|dbj\|BAD20362.1\| polyprotein [Turnip mosaic virus] | 400 | e−110 |
| gi\|47563789\|dbj\|BAD20354.1\| polyprotein [Turnip mosaic virus] | 400 | e−110 |
| gi\|47563855\|dbj\|BAD20387.1\| polyprotein [Turnip mosaic virus] | 400 | e−110 |
| gi\|47563803\|dbj\|BAD20361.1\| polyprotein [Turnip mosaic virus] | 400 | e−110 |
| gi\|47563841\|dbj\|BAD20380.1\| polyprotein [Turnip mosaic virus] | 400 | e−110 |
| gi\|51556046\|dbj\|BAD38766.1\| polyprotein [Turnip mosaic virus] | 399 | e−110 |
| gi\|18621213\|emb\|CAC87085.1\| polyprotein [Scallion mosaic virus] . . . | 399 | e−110 |
| gi\|51555984\|dbj\|BAD38735.1\| polyprotein [Turnip mosaic virus] | 399 | e−110 |
| gi\|51556034\|dbj\|BAD38760.1\| polyprotein [Turnip mosaic virus] | 399 | e−110 |
| gi\|47563869\|dbj\|BAD20394.1\| polyprotein [Turnip mosaic virus] | 399 | e−110 |
| gi\|47563871\|dbj\|BAD20395.1\| polyprotein [Turnip mosaic virus] | 399 | e−110 |
| gi\|51555980\|dbj\|BAD38733.1\| polyprotein [Turnip mosaic virus] | 399 | e−110 |
| gi\|51556012\|dbj\|BAD38749.1\| polyprotein [Turnip mosaic virus] | 399 | e−110 |
| gi\|51556008\|dbj\|BAD38747.1\| polyprotein [Turnip mosaic virus] | 399 | e−110 |
| gi\|4433369\|dbj\|BAA20962.1\| polyprotein [Soybean mosaic virus] | 399 | e−110 |
| gi\|51556036\|dbj\|BAD38761.1\| polyprotein [Turnip mosaic virus] | 399 | e−110 |
| gi\|94429\|pir∥JU0354 genome polyprotein - soybean mosaic virus ( . . . | 399 | e−110 |
| gi\|47563879\|dbj\|BAD20399.1\| polyprotein [Turnip mosaic virus] | 399 | e−110 |
| gi\|51556020\|dbj\|BAD38753.1\| polyprotein [Turnip mosaic virus] >g . . . | 399 | e−110 |
| gi\|51556038\|dbj\|BAD38762.1\| polyprotein [Turnip mosaic virus] | 399 | e−110 |
| gi\|9844585\|emb\|CAC03987.1\| polyprotein [Lettuce mosaic virus] | 399 | e−110 |
| gi\|47563833\|dbj\|BAD20376.1\| polyprotein [Turnip mosaic virus] | 399 | e−110 |
| gi\|51556072\|dbj\|BAD38779.1\| polyprotein [Turnip mosaic virus] >g . . . | 399 | e−110 |
| gi\|51555992\|dbj\|BAD38739.1\| polyprotein [Turnip mosaic virus] | 399 | e−110 |
| gi\|51556014\|dbj\|BAD38750.1\| polyprotein [Turnip mosaic virus] | 399 | e−110 |
| gi\|51556042\|dbj\|BAD38764.1\| polyprotein [Turnip mosaic virus] | 399 | e−110 |
| gi\|47563809\|dbj\|BAD20364.1\| polyprotein [Turnip mosaic virus] | 399 | e−110 |
| gi\|51556078\|dbj\|BAD38782.1\| polyprotein [Turnip mosaic virus] | 399 | e−110 |
| gi\|51556044\|dbj\|BAD38765.1\| polyprotein [Turnip mosaic virus] | 399 | e−110 |
| gi\|47563857\|dbj\|BAD20388.1\| polyprotein [Turnip mosaic virus] | 399 | e−110 |
| gi\|47563859\|dbj\|BAD20389.1\| polyprotein [Turnip mosaic virus] | 399 | e−110 |
| gi\|47563835\|dbj\|BAD20377.1\| polyprotein [Turnip mosaic virus] | 399 | e−110 |
| gi\|51556076\|dbj\|BAD38781.1\| polyprotein [Turnip mosaic virus] >g . . . | 399 | e−110 |
| gi\|47563825\|dbj\|BAD20372.1\| polyprotein [Turnip mosaic virus] | 399 | e−110 |
| gi\|47563797\|dbj\|BAD20358.1\| polyprotein [Turnip mosaic virus] | 399 | e−110 |
| gi\|47563885\|dbj\|BAD20402.1\| polyprotein [Turnip mosaic virus] | 399 | e−110 |
| gi\|9864421\|emb\|CAA66280.2\| polyprotein [Lettuce mosaic virus] >g . . . | 398 | e−110 |
| gi\|47563845\|dbj\|BAD20382.1\| polyprotein [Turnip mosaic virus] | 398 | e−110 |
| gi\|47563861\|dbj\|BAD20390.1\| polyprotein [Turnip mosaic virus] | 398 | e−110 |
| gi\|47563821\|dbj\|BAD20370.1\| polyprotein [Turnip mosaic virus] | 398 | e−110 |
| gi\|47563801\|dbj\|BAD20360.1\| polyprotein [Turnip mosaic virus] | 398 | e−110 |
| gi\|28193445\|emb\|CAC83742.1\| polyprotein [Lettuce mosaic virus] | 398 | e−110 |
| gi\|47563839\|dbj\|BAD20379.1\| polyprotein [Turnip mosaic virus] | 398 | e−110 |
| gi\|51556032\|dbj\|BAD38759.1\| polyprotein [Turnip mosaic virus] | 398 | e−110 |
| gi\|47563867\|dbj\|BAD20393.1\| polyprotein [Turnip mosaic virus] | 398 | e−110 |
| gi\|47563799\|dbj\|BAD20359.1\| polyprotein [Turnip mosaic virus] | 398 | e−110 |
| gi\|47563837\|dbj\|BAD20378.1\| polyprotein [Turnip mosaic virus] | 398 | e−110 |
| gi\|47563815\|dbj\|BAD20367.1\| polyprotein [Turnip mosaic virus] | 398 | e−110 |
| gi\|47563851\|dbj\|BAD20385.1\| polyprotein [Turnip mosaic virus] | 398 | e−110 |
| gi\|47563819\|dbj\|BAD20369.1\| polyprotein [Turnip mosaic virus] | 398 | e−110 |
| gi\|51556080\|dbj\|BAD38783.1\| polyprotein [Turnip mosaic virus] | 398 | e−110 |
| gi\|47563793\|dbj\|BAD20356.1\| polyprotein [Turnip mosaic virus] | 398 | e−110 |
| gi\|47563847\|dbj\|BAD20383.1\| polyprotein [Turnip mosaic virus] | 398 | e−110 |
| gi\|47563865\|dbj\|BAD20392.1\| polyprotein [Turnip mosaic virus] | 398 | e−110 |
| gi\|51556090\|dbj\|BAD38788.1\| polyprotein [Turnip mosaic virus] | 398 | e−110 |
| gi\|47563787\|dbj\|BAD20353.1\| polyprotein [Turnip mosaic virus] | 398 | e−110 |
| gi\|47563813\|dbj\|BAD20366.1\| polyprotein [Turnip mosaic virus] | 398 | e−110 |
| gi\|51556018\|dbj\|BAD38752.1\| polyprotein [Turnip mosaic virus] | 398 | e−110 |
| gi\|47563823\|dbj\|BAD20371.1\| polyprotein [Turnip mosaic virus] | 398 | e−110 |
| gi\|51556066\|dbj\|BAD38776.1\| polyprotein [Turnip mosaic virus] | 398 | e−110 |
| gi\|47563853\|dbj\|BAD20386.1\| polyprotein [Turnip mosaic virus] | 398 | e−110 |
| gi\|51556010\|dbj\|BAD38748.1\| polyprotein [Turnip mosaic virus] | 397 | e−110 |
| gi\|51555994\|dbj\|BAD38740.1\| polyprotein [Turnip mosaic virus] | 397 | e−110 |
| gi\|47563863\|dbj\|BAD20391.1\| polyprotein [Turnip mosaic virus] | 397 | e−110 |
| gi\|51556026\|dbj\|BAD38756.1\| polyprotein [Turnip mosaic virus] | 397 | e−110 |
| gi\|51556088\|dbj\|BAD38787.1\| polyprotein [Turnip mosaic virus] | 397 | e−110 |
| gi\|47563817\|dbj\|BAD20368.1\| polyprotein [Turnip mosaic virus] | 397 | e−109 |
| gi\|51556030\|dbj\|BAD38758.1\| polyprotein [Turnip mosaic virus] | 397 | e−109 |
| gi\|47563827\|dbj\|BAD20373.1\| polyprotein [Turnip mosaic virus] | 397 | e−109 |
| gi\|37731827\|gb\|AAO62574.1\| polyprotein [Plum pox virus] | 397 | e−109 |
| gi\|51556002\|dbj\|BAD38744.1\| polyprotein [Turnip mosaic virus] | 397 | e−109 |
| gi\|75616\|pir∥GNVSPD genome polyprotein - plum pox virus (strain D) | 397 | e−109 |
| gi\|28273148\|gb\|AAO38431.1\| polyprotein [Plum pox virus] | 397 | e−109 |
| gi\|28273150\|gb\|AAO38432.1\| polyprotein [Plum pox virus] | 397 | e−109 |
| gi\|1743376\|emb\|CAA34437.1\| unnamed protein product [Plum pox vir . . . | 397 | e−109 |
| gi\|25013638\|ref\|NP_734212.1\| NIa-Pro protein [Tobacco etch virus] | 397 | e−109 |
| gi\|9626509\|ref\|NP_040807.1\| polyprotein [Plum pox virus] >gi\|756 . . . | 397 | e−109 |
| gi\|51555996\|dbj\|BAD38741.1\| polyprotein [Turnip mosaic virus] | 397 | e−109 |
| gi\|47563811\|dbj\|BAD20365.1\| polyprotein [Turnip mosaic virus] | 397 | e−109 |
| gi\|51556004\|dbj\|BAD38745.1\| polyprotein [Turnip mosaic virus] | 397 | e−109 |
| gi\|51556082\|dbj\|BAD38784.1\| polyprotein [Turnip mosaic virus] | 397 | e−109 |
| gi\|531732\|emb\|CAA56974.1\| coat protein [Plum pox virus] >gi\|6284 . . . | 396 | e−109 |
| gi\|51556024\|dbj\|BAD38755.1\| polyprotein [Turnip mosaic virus] | 396 | e−109 |
| gi\|16075313\|emb\|CAC83052.1\| polyprotein [Dasheen mosaic virus] >. . . | 396 | e−109 |
| gi\|51556022\|dbj\|BAD38754.1\| polyprotein [Turnip mosaic virus] | 396 | e−109 |
| gi\|51556048\|dbj\|BAD38767.1\| polyprotein [Turnip mosaic virus] | 396 | e−109 |
| gi\|51555978\|dbj\|BAD38732.1\| polyprotein [Turnip mosaic virus] | 396 | e−109 |
| gi\|49659681\|gb\|AAK21975.2\| polyprotein [Plum pox virus] | 396 | e−109 |
| gi\|51556000\|dbj\|BAD38743.1\| polyprotein [Turnip mosaic virus] | 396 | e−109 |
| gi\|51556056\|dbj\|BAD38771.1\| polyprotein [Turnip mosaic virus] | 395 | e−109 |
| gi\|51555988\|dbj\|BAD38737.1\| polyprotein [Turnip mosaic virus] | 395 | e−109 |
| gi\|47563831\|dbj\|BAD20375.1\| polyprotein [Turnip mosaic virus] | 395 | e−109 |
| gi\|51555990\|dbj\|BAD38738.1\| polyprotein [Turnip mosaic virus] >g . . . | 395 | e−109 |
| gi\|32959776\|emb\|CAD56800.1\| polyprotein [Zucchini yellow mosaic . . . | 395 | e−109 |
| gi\|33391221\|gb\|AAQ17214.1\| polyprotein [Zucchini yellow mosaic v . . . | 395 | e−109 |
| gi\|51556058\|dbj\|BAD38772.1\| polyprotein [Turnip mosaic virus] | 395 | e−109 |
| gi\|37778798\|gb\|AAO61299.1\| polyprotein [Zucchini yellow mosaic v . . . | 395 | e−109 |
| gi\|32959936\|emb\|CAC87635.2\| polyprotein [Zucchini yellow mosaic . . . | 395 | e−109 |
| gi\|17059638\|ref\|NP_477522.1\| polyprotein [Zucchini yellow mosaic . . . | 395 | e−109 |
| gi\|33391223\|gb\|AAQ17215.1\| polyprotein [Zucchini yellow mosaic v . . . | 395 | e−109 |
| gi\|33391225\|gb\|AAQ17216.1\| polyprotein [Zucchini yellow mosaic v . . . | 395 | e−109 |
| gi\|418713\|pir∥GNVSRA genome polyprotein - plum pox virus (strai . . . | 395 | e−109 |
| gi\|51556084\|dbj\|BAD38785.1\| polyprotein [Turnip mosaic virus] | 395 | e−109 |
| gi\|51556060\|dbj\|BAD38773.1\| polyprotein [Turnip mosaic virus] | 395 | e−109 |
| gi\|32959938\|emb\|CAC87636.2\| polyprotein [Zucchini yellow mosaic . . . | 395 | e−109 |
| gi\|51556062\|dbj\|BAD38774.1\| polyprotein [Turnip mosaic virus] | 395 | e−109 |
| gi\|51556068\|dbj\|BAD38777.1\| polyprotein [Turnip mosaic virus] | 395 | e−109 |
| gi\|47563843\|dbj\|BAD20381.1\| polyprotein [Turnip mosaic virus] | 395 | e−109 |
| gi\|51556064\|dbj\|BAD38775.1\| polyprotein [Turnip mosaic virus] | 395 | e−109 |
| gi\|32959934\|emb\|CAC85170.2\| polyprotein [Zucchini yellow mosaic . . . | 394 | e−109 |
| gi\|25013919\|ref\|NP_734356.1\| NIa-Pro protein [Bean common mosaic . . . | 394 | e−108 |
| gi\|13940782\|gb\|AAB72004.2\| polyprotein [Zucchini yellow mosaic v . . . | 393 | e−108 |
| gi\|51555998\|dbj\|BAD38742.1\| polyprotein [Turnip mosaic virus] | 393 | e−108 |
| gi\|25013656\|ref\|NP_734220.1\| NIa-Pro protein [Turnip mosaic virus] | 393 | e−108 |
| gi\|51556054\|dbj\|BAD38770.1\| polyprotein [Turnip mosaic virus] | 393 | e−108 |
| gi\|847803\|gb\|AAA89116.1\| nuclear inclusion protein a | 393 | e−108 |
| gi\|19849802\|emb\|CAD22062.1\| polyprotein [Zucchini yellow mosaic . . . | 393 | e−108 |
| gi\|51556052\|dbj\|BAD38769.1\| polyprotein [Turnip mosaic virus] | 393 | e−108 |
| gi\|51556050\|dbj\|BAD38768.1\| polyprotein [Turnip mosaic virus] | 393 | e−108 |
| gi\|51556086\|dbj\|BAD38786.1\| polyprotein [Turnip mosaic virus] | 393 | e−108 |
| gi\|9633629\|ref\|NP_051161.1\| polyprotein [Japanese yam mosaic vir . . . | 393 | e−108 |
| gi\|25013526\|ref\|NP_734386.1\| NIa-Pro protein [Cowpea aphid-borne . . . | 391 | e−108 |
| gi\|466348\|gb\|AAA65559.1\| polyprotein >gi\|3915808\|sp\|P18479\|POLG_. . . | 391 | e−108 |
| gi\|51556006\|dbj\|BAD38746.1\| polyprotein [Turnip mosaic virus] | 391 | e−108 |
| gi\|938312\|emb\|CAA48521.1\| unnamed protein product [Zucchini yell . . . | 391 | e−108 |
| gi\|25013496\|ref\|NP_734120.1\| NIa-Pro protein [Bean common mosaic . . . | 390 | e−107 |
| gi\|5650730\|emb\|CAB51641.1\| polyprotein [Plum pox virus] | 390 | e−107 |
| gi\|294314\|gb\|AAB05823.1\| polyprotein [Plum pox virus] >gi\|391441 . . . | 390 | e−107 |
| gi\|13235336\|emb\|CAB75857.2\| polyprotein [Potato virus V] >gi\|214 . . . | 390 | e−107 |
| gi\|21464610\|emb\|CAD28624.1\| polyprotein [Potato virus Y] | 389 | e−107 |
| gi\|51949954\|ref\|YP_077275.1\| protease [Watermelon mosaic virus] | 389 | e−107 |
| gi\|4092844\|dbj\|BAA36278.1\| polyprotein [Japanese yam mosaic virus] | 389 | e−107 |
| gi\|21464608\|emb\|CAD28623.1\| polyprotein [Potato virus Y] | 389 | e−107 |
| gi\|27371970\|gb\|AAN87844.1\| polyprotein [Potato virus Y strain N] | 388 | e−107 |
| gi\|19716316\|gb\|AAL95713.1\| polyprotein [Potato virus Y] | 387 | e−106 |
| gi\|420750\|pir∥JN0545 genome polyprotein - potato virus Y (isola . . . | 387 | e−106 |
| gi\|1430930\|emb\|CAA66472.1\| polyprotein [Potato virus Y] | 387 | e−106 |
| gi\|27371968\|gb\|AAN87843.1\| polyprotein [Potato virus Y strain NTN] | 387 | e−106 |
| gi\|25013780\|ref\|NP_734316.1\| NIa-Pro protein [Sweet potato feath . . . | 387 | e−106 |
| gi\|54021379\|emb\|CAE51230.1\| polyprotein [Potato virus Y] | 386 | e−106 |
| gi\|6066611\|emb\|CAB58238.1\| polyprotein [Potato virus A] | 386 | e−106 |
| gi\|333304\|gb\|AAA47085.1\| ORF | 386 | e−106 |
| gi\|23477610\|gb\|AAN34778.1\| polyprotein [Potato virus A] | 386 | e−106 |
| gi\|23955468\|gb\|AAN40503.1\| polyprotein [Potato virus A] | 386 | e−106 |
| gi\|18621163\|emb\|CAC84095.1\| polyprotein [Sugarcane mosaic virus] | 386 | e−106 |
| gi\|39163615\|ref\|NP_945133.1\| polyprotein [Lily mottle virus] >gi . . . | 386 | e−106 |
| gi\|6066615\|emb\|CAB58240.1\| polyprotein [Potato virus A] | 386 | e−106 |
| gi\|53913355\|emb\|CAE51192.1\| polyprotein [Potato virus Y] | 386 | e−106 |
| gi\|53913357\|emb\|CAE51193.1\| polyprotein [Potato virus Y] | 386 | e−106 |
| gi\|25013618\|ref\|NP_734202.1\| NIa-Pro protein [Soybean mosaic virus] | 386 | e−106 |
| gi\|11414847\|emb\|CAC17411.1\| polyprotein [Potato virus A] >gi\|214 . . . | 385 | e−106 |
| gi\|53749596\|emb\|CAE50910.1\| polyprotein [Potato virus Y] | 385 | e−106 |
| gi\|53850822\|gb\|AAU95465.1\| polyprotein [Potato virus Y] | 385 | e−106 |
| gi\|53850824\|gb\|AAU95466.1\| polyprotein [Potato virus Y] | 385 | e−106 |
| gi\|6066617\|emb\|CAB58241.1\| polyprotein [Potato virus A] | 385 | e−106 |
| gi\|53913351\|emb\|CAE51190.1\| polyprotein [Potato virus Y] | 384 | e−106 |
| gi\|130497\|sp\|P20234\|POLG_OMV Genome polyprotein [Contains: Nucle . . . | 384 | e−105 |
| g\|18621157\|emb\|CAC84092.1\| polyprotein [Sugarcane mosaic virus] | 383 | e−105 |
| gi\|45774602\|gb\|AAS76887.1\| polyprotein [Sugarcane mosaic virus] | 383 | e−105 |
| gi\|18621159\|emb\|CAC84093.1\| polyprotein [Sugarcane mosaic virus] | 383 | e−105 |
| gi\|27528455\|emb\|CAC81986.1\| polyprotein [Sugarcane mosaic virus] | 383 | e−105 |
| gi\|1906388\|gb\|AAB50573.1\| polyprotein [Potato virus Y] | 383 | e−105 |
| gi\|18621203\|emb\|CAC84438.1\| polyprotein [Sorghum mosaic virus] | 383 | e−105 |
| gi\|441194\|dbj\|BAA00342.1\| polyprotein [Potato virus Y] >gi\|13467 . . . | 383 | e−105 |
| gi\|77389\|pir∥JS0166 genome polyprotein - potato virus Y (strain N) | 383 | e−105 |
| gi\|21913302\|gb\|AAM81207.1\| polyprotein [Potato virus Y] >gi\|9627 . . . | 383 | e−105 |
| gi\|53913353\|emb\|CAE51191.1\| polyprotein [Potato virus Y] | 383 | e−105 |
| gi\|20270986\|gb\|AAM18491.1\| polyprotein [Sugarcane mosaic virus] | 382 | e−105 |
| gi\|25013604\|ref\|NP_734248.1\| NIa-Pro protein [Potato virus Y] | 371 | e−102 |
| gi\|18490053\|ref\|NP_569138.1\| polyprotein [Maize dwarf mosaic vir . . . | 370 | e−101 |
| gi\|25013626\|ref\|NP_734140.1\| NIa-Pro protein [Sugarcane mosaic v . . . | 370 | e−101 |
| gi\|48249206\|ref\|YP_022761.1\| NIa-Pro protein [Yam mosaic virus] | 369 | e−101 |
| gi\|39163623\|ref\|NP_945143.1\| NIa-Pro [Lily mottle virus] | 369 | e−101 |
| gi\|25013596\|ref\|NP_734366.1\| NIa-Pro protein [Potato virus A] | 368 | e−101 |
| gi\|25013578\|ref\|NP_734438.1\| NIa-Pro protein [Pepper mottle virus] | 368 | e−101 |
| gi\|27519887\|gb\|AAB70862.2\| polyprotein [Sorghum mosaic virus] | 366 | e−100 |
| gi\|32490549\|ref\|NP_870995.1\| polyprotein [Papaya leaf-distortion . . . | 366 | e−100 |
| gi\|25013838\|ref\|NP_734415.1\| NIa-Pro protein [Peanut mottle virus] | 365 | e−100 |
| gi\|40254036\|ref\|NP_954626.1\| NIa-Pro [Beet mosaic virus] | 364 | e−100 |
| gi\|1944183\|dbj\|BAA19654.1\| polyprotein [Clover yellow vein virus] | 364 | e−100 |
| gi\|20087031\|ref\|NP_613273.1\| polyprotein [Clover yellow vein vir . . . | 364 | e−100 |
| gi\|1054945\|gb\|AAB52962.1\| polyprotein | 363 | 1e−99 |
| gi\|29611981\|ref\|NP_818992.1\| NIa-Pro protein [Peru tomato mosaic . . . | 363 | 2e−99 |
| gi\|45004663\|ref\|NP_982342.1\| nuclear inclusion protein A [Chilli . . . | 363 | 3e−99 |
| gi\|25013899\|ref\|NP_734100.1\| NIa-Pro protein [Leek yellow stripe . . . | 362 | 3e−99 |
| gi\|29611980\|ref\|NP_818993.1\| NIa-Pro protein [Wild potato mosaic . . . | 361 | 7e−99 |
| gi\|25013546\|ref\|NP_734150.1\| NIa-Pro (Nuclear inclusion protein, . . . | 361 | 7e−99 |
| gi\|2598610\|emb\|CAA27720.1\| polyprotein [Tobacco vein mottling vi . . . | 360 | 1e−98 |
| gi\|75614\|pir∥GNVSTV genome polyprotein - tobacco vein mottling . . . | 360 | 1e−98 |
| gi\|18621201\|emb\|CAC84437.1\| polyprotein [Sorghum mosaic virus] >. . . | 360 | 2e−98 |
| gi\|32493295\|ref\|NP_871745.1\| NIa-Pro protein [Onion yellow dwarf . . . | 358 | 8e−98 |
| gi\|555290\|gb\|AAA91583.1\| polyprotein | 356 | 3e−97 |
| gi\|7414435\|emb\|CAB85904.1\| putative L1 polyprotein [Pea seed-bor . . . | 354 | 8e−97 |
| gi\|6911259\|gb\|AAF31455.1\|nuclear inclusion protein A [Tobacco v . . . | 353 | 2e−96 |
| gi\|9628430\|ref\|NP_056765.1\| polyprotein [Pea seed-borne mosaic v . . . | 350 | 1e−95 |
| gi\|32493285\|ref\|NP_871735.1\| NIa-Pro [Papaya leaf-distortion mos . . . | 350 | 2e−95 |
| gi\|25013516\|ref\|NP_734170.1\| NIa-Pro protein [Clover yellow vein . . . | 349 | 3e−95 |
| gi\|975217\|emb\|CAA62014.1\| polyprotein [Pea seed-borne mosaic virus] | 349 | 4e−95 |
| gi\|61351\|emb\|CAA47905.1\| polyprotein [Papaya ringspot virus] >gi . . . | 348 | 7e−95 |
| gi\|25013644\|ref\|NP_734334.1\| NIa-Pro protein [Tobacco vein mottl . . . | 347 | 1e−94 |
| gi\|1354082\|gb\|AAB37237.1\| polyprotein | 347 | 2e−94 |
| gi\|25013829\|ref\|NP_734090.1\| NIa-Pro protein [Sorghum mosaic virus] | 346 | 2e−94 |
| gi\|559367\|dbj\|BAA05979.1\| polypeptide [Bean yellow mosaic virus] | 346 | 2e−94 |
| gi\|19881395\|ref\|NP_612218.1\| polyprotein [Bean yellow mosaic vir . . . | 346 | 2e−94 |
| gi\|29469900\|gb\|AAO74620.1\| polyprotein [Papaya ringspot virus] | 346 | 3e−94 |
| gi\|20336646\|gb\|AAM19343.1\| polyprotein [Cocksfoot streak virus] . . . | 346 | 3e−94 |
| gi\|37780264\|gb\|AAP30002.1\| polyprotein [Bean yellow mosaic virus] | 345 | 4e−94 |
| gi\|1771471\|emb\|CAA65886.1\| PRSV YK polyprotein [Papaya ringspot . . . | 343 | 2e−93 |
| gi\|94419\|pir∥S18921 genome polyprotein - bean yellow mosaic vir . . . | 343 | 2e−93 |
| gi\|312734\|emb\|CAA44960.1\| unnamed protein product [Bean yellow m . . . | 343 | 2e−93 |
| gi\|27542792\|gb\|AAO16605.1\| polyprotein [Papaya ringspot virus] | 343 | 2e−93 |
| gi\|23680942\|gb\|AAK17011.2\| polyprotein [Papaya ringspot virus W] | 342 | 3e−93 |
| gi\|25013566\|ref\|NP_734426.1\| NIa-Pro protein [Pea seed-borne mos . . . | 341 | 7e−93 |
| gi\|14581405\|gb\|AAG47346.1\| polyprotein [Papaya ringspot virus W] | 339 | 3e−92 |
| gi\|1724087\|gb\|AAB38493.1\| nuclear inclusion A [bean yellow mosai . . . | 336 | 2e−91 |
| gi\|25013556\|ref\|NP_734240.1\| NIa-Pro protein [Papaya ringspot vi . . . | 334 | 1e−90 |
| gi\|25014045\|ref\|NP_734396.1\| NIa-Pro protein [Cocksfoot streak v . . . | 333 | 2e−90 |
| gi\|25013506\|ref\|NP_734180.1\| NIa-Pro protein [Bean yellow mosaic . . . | 333 | 2e−90 |
| gi\|48843531\|ref\|YP_025106.1\| polyprotein [Agropyron mosaic virus . . . | 332 | 4e−90 |
| gi\|48843533\|ref\|YP_025107.1\| polyprotein [Hordeum mosaic virus] . . . | 329 | 3e−89 |
| gi\|20153408\|ref\|NP_619668.1\| polyprotein [Johnsongrass mosaic vi . . . | 323 | 2e−87 |
| gi\|2145465\|emb\|CAA70983.1\| polyprotein [Ryegrass mosaic virus]>. . . | 320 | 2e−86 |
| gi\|25013866\|ref\|NP_734326.1\| NIa-Pro protein [Ryegrass mosaic vi . . . | 319 | 4e−86 |
| gi\|51101435\|ref\|YP_063393.1\| NIa-Pro [Hordeum mosaic virus] | 318 | 7e−86 |
| gi\|50404820\|ref\|YP_054399.1\| NIa-Pro [Agropyron mosaic virus] | 317 | 1e−85 |
| gi\|3282654\|gb\|AAC25028.1\| polyprotein [Ryegrass mosaic virus] | 315 | 5e−85 |
| gi\|3873620\|emb\|CAA10100.1\| polyprotein [Bean common mosaic virus] | 313 | 2e−84 |
| gi\|25013815\|ref\|NP_734405.1\| NIa-Pro protein [Johnsongrass mosai . . . | 311 | 1e−83 |
| gi\|497916\|gb\|AAB50167.1\| polyprotein | 306 | 2e−82 |
| gi\|41411200\|emb\|CAF22057.1\| polyprotein [Bean yellow mosaic virus] | 292 | 4e−78 |
| gi\|28194134\|gb\|AAO33413.1\| polyprotein precursor [Zantedeschia s . . . | 253 | 3e−66 |
| gi\|130527\|sp\|P18478\|POLG_WMV2U Genome polyprotein [Contains: Nuc.. | 252 | 7e−66 |
| gi\|2952295\|gb\|AAC05494.1\| polyprotein [Dasheen mosaic virus] | 225 | 8e−58 |
| gi\|1771709\|emb\|CAA97466.1\| polyprotein [Sweet potato mild mottle . . . | 199 | 3e−50 |
| gi\|25013880\|ref\|NP_734291.1\| NIa-Pro protein [Sweet potato mild . . . | 196 | 3e−49 |
| gi\|994796\|emb\|CAA88417.1\| polyprotein [Brome streak mosaic virus . . . | 185 | 5e−46 |
| gi\|25013909\|ref\|NP_734260.1\| NIa-Pro protein [Brome streak mosai . . . | 184 | 2e−45 |
| gi\|39103366\|emb\|CAE83574.1\| polyprotein [Vanilla mosaic virus] | 171 | 1e−41 |
| gi\|11066856\|gb\|AAG28732.1\| polyprotein [Wheat streak mosaic virus] | 168 | 9e−41 |
| gi\|37651480\|ref\|NP_932608.1\| polyprotein [Oat necrotic mottle vi . . . | 168 | 1e−40 |
| gi\|221426\|dbj\|BAA01892.1\| polyprotein precursor [Leek yellow str . . . | 168 | 1e−40 |
| gi\|11066854\|gb\|AAG28731.1\| polyprotein [Wheat streak mosaic virus] | 166 | 4e−40 |
| gi\|38304208\|ref\|NP_940829.1\| NIa-Pro protein [Oat necrotic mottl . . . | 165 | 6e−40 |
| gi\|3047321\|gb\|AAC13692.1\| polyprotein [Wheat streak mosaic virus . . . | 165 | 9e−40 |
| gi\|13241968\|gb\|AAK16492.1\| polyprotein [Expression vector pWSMV- . . . | 165 | 9e−40 |
| gi\|17981494\|gb\|AAL51041.1\| polyprotein [Wheat streak mosaic virus] | 165 | 9e−40 |
| gi\|17981492\|gb\|AAL51040.1\| polyprotein [Wheat streak mosaic virus] | 165 | 1e−39 |
| gi\|25013806\|ref\|NP_734272.1\| NIa-Pro protein [Wheat streak mosai . . . | 164 | 1e−39 |
| gi\|2197108\|gb\|AAC58509.1\| polyprotein [Tobacco etch virus] | 160 | 2e−38 |
| gi\|9558714\|gb\|AAB29948.2\| polyprotein [Sweet potato feathery mot . . . | 151 | 2e−35 |
| gi\|19744020\|emb\|CAA76842.3\| polyprotein [Sugarcane streak mosaic . . . | 150 | 2e−35 |
| gi\|2554632\|dbj\|BAA22880.1\| polyprotein [Bean yellow mosaic virus] | 145 | 6e−34 |
| gi\|49182260\|gb\|AAT57632.1\| polyprotein [Sugarcane mosaic virus] | 145 | 1e−33 |
| gi\|499030\|emb\|CAA52087.1\| unnamed protein product [Wheat spindle . . . | 144 | 1e−33 |
| gi\|575957\|emb\|CAA55300.1\| coat protein; nuclar inclusion protein . . . | 128 | 1e−28 |
| gi\|3218532\|dbj\|BAA28768.1\| polyprotein [Wheat yellow mosaic viru . . . | 121 | 1e−26 |
| gi\|6272288\|emb\|CAB60138.1\| putative polyprotein [Wheat yellow mo . . . | 120 | 2e−26 |
| gi\|6137095\|emb\|CAB59644.1\| polyprotein [Wheat yellow mosaic virus] | 119 | 6e−26 |
| gi\|25013963\|ref\|NP_697038.1\| NIa-Pro protein [Wheat yellow mosai . . . | 118 | 9e−26 |
| gi\|34582181\|emb\|CAD56475.1\| polyprotein 1 [Barley yellow mosaic . . . | 116 | 4e−25 |
| gi\|853780\|emb\|CAA55237.1\| viral polymerase, coat protein [Brome . . . | 115 | 8e−25 |
| gi\|34582175\|emb\|CAD56472.1\| polyprotein 1 [Barley yellow mosaic . . . | 114 | 2e−24 |
| gi\|34582179\|emb\|CAD56474.1\| polyprotein 1 [Barley yellow mosaic . . . | 114 | 2e−24 |
| gi\|34582185\|emb\|CAD56477.1\| polyprotein 1 [Barley yellow mosaic . . . | 114 | 2e−24 |
| gi\|58679\|emb\|CAA49412.1\| C1 (helicase); NIa (proteinase); NIb (r . . . | 114 | 2e−24 |
| gi\|34582183\|emb\|CAD56476.1\| polyprotein 1 [Barley yellow mosaic . . . | 112 | 6e−24 |
| gi\|34582173\|emb\|CAD56471.1\| polyprotein 1 [Barley yellow mosaic . . . | 112 | 7e−24 |
| gi\|11559225\|dbj\|BAB18744.1\| 270 K polyprotein [Barley yellow mosa . . . | 110 | 3e−23 |
| gi\|450361\|emb\|CAA82642.1\| polyprotein [Potato virus Y] | 110 | 3e−23 |
| gi\|34582177\|emb\|CAD56473.1\| polyprotein 1 [Barley yellow mosaic . . . | 108 | 1e−22 |
| gi\|21427655\|ref\|NP_659025.1\| polyprotein [Oat mosaic virus] >gi\|. . . | 105 | 1e−21 |
| gi\|25014011\|ref\|NP_734280.1\| NIa-Pro protein [Oat mosaic virus] | 103 | 5e−21 |
| gi\|5596368\|gb\|AAD45560.1\| 270 kDa precursor protein [wheat yello . . . | 102 | 7e−21 |
| gi\|221110\|dbj\|BAA00875.1\| polyprotein [Barley yellow mosaic viru . . . | 97 | 4e−19 |
| gi\|5019292\|emb\|CAB44430.1\| RNA1 polyprotein [Barley mild mosaic . . . | 96 | 6e−19 |
| gi\|15808066\|ref\|NP_148999.1\| polyprotein [Barley yellow mosaic v . . . | 96 | 7e−19 |
| gi\|51241614\|emb\|CAD66659.1\| polyprotein [Barley mild mosaic virus] | 95 | 9e−19 |
| gi\|51241616\|emb\|CAD66660.1\| polyprotein [Barley mild mosaic virus] | 95 | 9e−19 |
| gi\|51241612\|emb\|CAD66658.1\| polyprotein [Barley mild mosaic virus] | 95 | 9e−19 |
| gi\|33331076\|gb\|AAQ10774.1\| polyprotein [Barley yellow mosaic virus] | 95 | 1e−18 |
| gi\|1181180\|dbj\|BAA01742.1\| polyprotein precursor [Barley mild mo . . . | 95 | 1e−18 |
| gi\|1339796\|gb\|AAC42215.1\| polyprotein | 95 | 2e−18 |
| gi\|2661743\|emb\|CAA71869.1\| RNA1 polyprotein [Barley mild mosaic . . . | 95 | 2e−18 |
| gi\|2661745\|emb\|CAA71870.1\| RNA1 polyprotein [Barley mild mosaic . . . | 95 | 2e−18 |
| gi\|321630\|pir∥PQ0440 polyprotein - barley mild mosaic virus (st . . . | 95 | 2e−18 |
| gi\|25013744\|ref\|NP_734306.1\| NIa-Pro protease [Barley yellow mos . . . | 94 | 2e−18 |
| gi\|25013752\|ref\|NP_734298.1\| NIa-Pro protein [Barley mild mosaic . . . | 91 | 2e−17 |
| gi\|33331044\|gb\|AAQ10758.1\| polyprotein [Barley mild mosaic virus] | 90 | 4e−17 |
| gi\|1905770\|dbj\|BAA18953.1\| polyprotein precursor [Barley mild mo . . . | 90 | 5e−17 |
| gi\|221058\|dbj\|BAA01741.1\| polyprotein precursor [Barley mild mos . . . | 89 | 6e−17 |
| gi\|419028\|pir∥A60678 genome polyprotein - potato virus Y (strai . . . | 83 | 4e−15 |
| gi\|29125692\|emb\|CAD79433.1\| polyprotein [Cardamom mosaic virus] | 80 | 4e−14 |
| gi\|53139449\|emb\|CAH59107.1\| VPg protein [Lily mottle virus] | 69 | 7e−11 |
| gi\|18483223\|gb\|AAL73971.1\| polyprotein [Sunflower mosaic virus] | 59 | 1e−07 |
| gi\|6996532\|emb\|CAB75431.1\| potyviral polypeptide [Potato virus A] | 58 | 1e−07 |
| gi\|9663833\|emb\|CAC01251.1\| potyviral polypeptide [Potato virus A] | 58 | 1e−07 |
| gi\|11066408\|gb\|AAG28576.1\| pol protein [Peanut stripe virus] | 52 | 9e−06 |

TABLE 2
Protease and recognition/cleavage site

| Protease | Recongition sequence/cleavage site |
|---|---|
| Enterokinase | Asp-Asp-Asp-Asp-Lys |
| Factor Xa protease | Ile-Glu/Asp-Gly-Arg |
| Thrombin | Leu-Val-Pro-Arg-Gly-Ser |
| TEV protease | Glu-Xaa-Xaa-Tyr-Xaa-Gln-Ser, where |
| | Xaa can be any amino acid residue |
| PreScission ™ protease | Leu-Glu-Val-Leu-Phe-Gln-Gly-Pro |
| TVMV protease | ETVRFQS |
| | Glu-Thr-Val-Arg-Phe-Glu-Ser |

Table 2 provides an exemplary list of proteases and their recognition sequences. Any highly specific protease whose recognition sequence is known is suitable for the compositions and methods disclosed herein.

### DOCUMENTS

The following documents are incorporated by reference to the extent they relate to or describe materials or methods disclosed herein.
Alexandrov A, Dutta K, Pascal S M. MBP fusion protein with a viral protease cleavage site: one-step cleavage/purification of insoluble proteins. Biotechniques. June 2001; 30(6):1194-8. No abstract available.
Donnelly, M. I., P. Wilkins Stevens, L. Stols, S. X. Su, S. Tollaksen, C. S. Giometti, and A. Joachimiak (2001) Expression of a Highly Toxic Protein, Bax, in Escherichia coli by Attachment of a Leader Peptide Derived from the GroES Co-chaperone. Protein Expr. Purif. 22, 422-429.
Fox J D, Waugh D S., Maltose-binding protein as a solubility enhancer. Methods Mol Biol. 2003; 205:99-117.
Hearn M T, Acosta D., Applications of novel affinity cassette methods: use of peptide fusion handles for the purification of recombinant proteins. J Mol Recognit. November-December 2001; 14(6):323-69.
Kapust, R. B. et al. Tobacco etch virus protease: mechanism of autolysis and rational design of stable mutants with wild-type catalytic proficiency. Protein Eng 14, 993-1000 (2001).
Kapust R B, Tozser J, Copeland T D, Waugh D S., The P1′ specificity of tobacco etch virus protease. Biochem Biophys Res Commun. Jun. 28, 2002; 294(5):949-55.
Kapust R B, Waugh D S., Controlled intracellular processing of fusion proteins by TEV protease. Protein Expr Purif. July 2000; 19(2):312-8.
Kapust R B, Waugh D S., Escherichia coli maltose-binding protein is uncommonly effective at promoting the solubility of polypeptides to which it is fused. Protein Sci. August 1999; 8(8):1668-74.
Kim, Y., I. Dementieva, M. Zhou, R. Wu, L. Lezondra, P. Quartey, G. Joachimiak, O. Korolev, H. Li, and A. Joachimiak, Automation of protein purification for structural genomics. J. Struct. Funct. Genomics 5:111-8, 2004.
Millard, C. S. et al. A less laborious approach to the high-throughput production of recombinant proteins in Escherichia coli using 2-liter plastic bottles. Protein Expr Purif 29, 311-320, 2003.
Nallamsetty, S. et al. Efficient site-specific processing of fusion proteins by tobacco vein mottling virus protease in vivo and in vitro. Protein Expr Purif 38, 108-115 (2004).
Phan J, Zdanov A, Evdokimov A G, Tropea J E, Peters H K 3rd, Kapust R B, Li M, Wlodawer A, Waugh D S., Structural basis for the substrate specificity of tobacco etch virus protease. J Biol Chem. Dec. 27, 2002; 277(52):50564-72. Epub Oct. 10, 2002.
Pryor K D, Leiting B., High-level expression of soluble protein in Escherichia coli using a His6-tag and maltose-binding-protein double-affinity fusion system. Protein Expr Purif. August 1997; 10(3):309-19.
Riggs P., Expression and purification of recombinant proteins by fusion to maltose-binding protein. Mol Biotechnol. May 2000; 15(1):51-63.
Routzahn K M, Waugh D S., Differential effects of supplementary affinity tags on the solubility of MBP fusion proteins. J Struct Funct Genomics. 2002; 2(2):83-92.
Sachdev D, Chirgwin J M., Solubility of proteins isolated from inclusion bodies is enhanced by fusion to maltose-binding protein or thioredoxin. Protein Expr Purif. February 1998; 12(1):122-32.
Stols, L., M. Gu, L. Dieckman, R. Raffen, F. R. Collart, and M. I. Donnelly, A new vector for high-throughput, ligation-independent cloning encoding a tobacco etch virus protease cleavage site. Protein Expr. Purif. 25:8-15, 2002.
Stols, L., C. S. Millard, I. Dementieva, and M. I. Donnelly, Production of selenomethionine-labeled proteins in two-liter plastic bottles for structure determination. J. Struct. Funct. Genomics 5:95-102, 2004.

1. A nucleic acid molecule comprising:
(a) a first nucleotide sequence encoding a first tag;
(b) a second nucleotide sequence encoding a second tag, wherein the second tag is a protein or a peptide that promotes affinity purification;
(c) a third nucleotide sequence encoding a first recognition peptide sequence for a first specific protease; and
(d) a fourth nucleotide sequence encoding a second recognition peptide sequence for a second specific protease.

2. The nucleic acid molecule of claim 1 further comprising a fifth nucleotide sequence encoding a target protein or a peptide.

3. The nucleic acid molecule of claim 1, wherein the first recognition sequence is positioned between the first tag and the second tag; and the second recognition sequence is downstream or upstream of the second tag.

4. The nucleic acid molecule of claim 1, wherein the first tag is a protein or a peptide that improves protein production expression, protein folding, protein solubility, or improves the combination thereof.

5. The nucleic acid molecule of claim 1, wherein the first tag is a maltose binding protein (MBP).

6. The nucleic acid molecule of claim 1, wherein the second tag is a poly-histidine.

7. The nucleic acid molecule of claim 6, wherein the first specific protease and the second specific protease are selected from the group consisting of proteases listed in TABLE I, and wherein the first specific protease and the second specific protease are distinct.

8. The nucleic acid molecule of claim 1, wherein the first specific protease is tobacco vein mottling virus (TVMV) protease, and the second specific protease is tobacco etch virus (TEV) protease.

9. A vector comprising the nucleic acid of claim 1.

10. A vector comprising the nucleic acid of claim 2.

11. A vector comprising the nucleic acid of claim 3.

12. A vector comprising the nucleic acid of claim 4.

13. A vector comprising the nucleic acid of claim 5.

14. A vector comprising the nucleic acid of claim 6.

15. A vector comprising the nucleic acid of claim 7.

16. A vector comprising the nucleic acid of claim 8.

17. A vector comprising a nucleotide sequence encoding a peptide sequence comprising N-helpertag-site1-markertag-site2-target,
wherein N is N-terminus of the peptide, helpertag is a protein or peptide for improving protein expression, folding or solubility, site1 is a first recognition peptide sequence that is cleaved by a first specific protease, marker tag is a peptide used for purification or detection; site2 is a second recognition peptide sequence that is cleaved by a second specific protease, and target is a protein or a peptide of interest.

18. A vector comprising a nucleotide sequence encoding a peptide sequence of comprising N-target-site2-markertag-site1-helpertag,
wherein N is N-terminus of the peptide, helpertag is a protein or peptide for improving protein expression, folding or solubility, site1 is a first recognition peptide sequence that is cleaved by a first specific protease, marker tag is a peptide sequence used for purification or detection; site2 is a second recognition peptide sequence that is cleaved by a second specific protease, and target is a protein or peptide of interest.

19. The vector of claim 17, wherein
the helpertag is a maltose binding protein (MBP), the site1 is a recognition peptide sequence that is cleaved by a tobacco vein mottling virus (TVMV) protease, the markertag is a poly-histidine tag (his₆; SEQ ID NO:14), the site2 is a recognition peptide sequence that is cleaved by a tobacco etch virus (TEV) protease and the target is a protein of interest.

20. A method of protein production, the method comprising:
(a) expressing a target protein in a cell using a vector comprising a nucleotide sequence encoding a peptide sequence of N-helpertag-site1-markertag-site2-target, or a nucleotide sequence encoding a peptide sequence of N-target-site2-markertag-site1-helpertag,
wherein N is N-terminus of the peptide, helpertag is a beneficial protein or peptide sequence for improving protein expression, folding or solubility, site1 is a recognition peptide sequence that is cleavable by a first specific enzyme, markertag is a peptide sequence used for purification, detection or other application; site2 is another recognition peptide sequence that is cleavable by a second specific enzyme, and target is a protein of interest;
(b) cleaving the encoded peptide at site1 with the first specific enzyme to produce a polypeptide of markertag-site2-target or target-site2-markertag;
(c) isolating the cleaved peptide of (b);
(d) cleaving the isolated peptide of (c) at site2 with the second specific enzyme to produce the target protein; and
(e) isolating the target protein.

21. The method of claim 20, further comprising co-expressing a gene encoding the first specific enzyme.

22. The method of claim 21, wherein the first specific enzyme cleaves the encoded peptide at site1 in vivo.

23. The method of claim 20, wherein the first specific enzyme and the second specific enzyme are distinct proteases.

24. The method of claim 23, wherein each distinct protease is selected from the list in TABLE I.

25. The method of claim 20, wherein the helpertag is a maltose binding protein (MBP), site1 is a peptide sequence recognized by the tobacco vein mottling virus (TVMV) protease, the markertag is a poly-histidine tag (his₆; SEQ ID NO:14), and site2 is a peptide sequence recognized by the tobacco etch virus (TEV) protease.

26. The method of claim 20, wherein the step (c) and (e) are performed using immobilized metal ion affinity chromatography (IMAC).

27. A method of producing a protein comprising:

28. (a) introducing a vector into a cell, wherein the vector comprises a nucleotide sequence encoding a peptide sequence of N-MBP-tvmv-his₆-tev-target (6×His tag disclosed as SEQ ID NO: 14), or N-target-tev-his₆-tvmv-MBP (6×His tag disclosed as SEQ ID NO: 14),
wherein N is N-terminus of the peptide, MBP is a maltose binding protein, tvmv is a recognition peptide sequence that is cleaved by a tobacco vein mottling virus (TVMV) protease, his₆ (SEQ ID NO: 14) is a poly-histidine tag, tev is a recognition peptide sequence that is cleaved by a the tobacco etch virus (TEV) protease, and target is a protein of interest;
(b) co-expressing a gene encoding TVMV protease;
(c) extracting protein from the cell;
(d) isolating the his₆-tev-target (6×His tag disclosed as SEQ ID NO: 14) or target-tev-his₆ peptide (6×His tag disclosed as SEQ ID NO: 14);
(e) treating the isolated peptide from (d) with the TEV protease; and
(f) isolating the target protein.

29. The method of claim 20, wherein the cell is selected from the group consisting of bacterial cells, insect cells, animal and plant cells.

30. A cell transformed with the nucleic acid molecule of claim 1.


SEQUENCE LISTING (14 sequences)

SEQ ID NO: 1 - DNA, 35 bp, Artificial Sequence
  Description of Artificial Sequence Synthetic primer
ttttagatctgatgtcccctatactaggttattgg

SEQ ID NO: 2 - DNA, 45 bp, Artificial Sequence
  Description of Artificial Sequence Synthetic primer
ttttggtacctgggatatcgtaatcatccgattttggaggatggt

SEQ ID NO: 3 - DNA, 6441 bp, Artificial Sequence
  Description of Artificial Sequence Synthetic pMCSG19 nucleotide sequence
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagtgcggccgcaagcttgtcgacggagctcgaattcggatccgttatccacttccaatattggattggaagtacaggttctcggtacccagatccacgccagaagagtgatgatgatggtggtgagactggaaacgcacggtttccgagcctgcttttttgtacaaacttgtgatcgaattagtctgcgcgtctttcagggcttcatcgacagtctgacgaccgctggcggcgttgatcaccgcagtacgcacggcataccagaaagcggacatctgcgggatgttcggcatgatttcacctttctgggcgttttccatggtggcggcaatacgtggatctttcgccaactcttcctcgtaagacttcagcgctacggcacccagcggtttgtctttattaaccgcttccagaccttcatcagtcagcagatagttttcgaggaactcttttgccagctctttgttcggactggcggcgttaatacctgcgctcagcacgccaacgaacggtttggatggttgacccttgaaggtcggcagtaccgttacaccataattcactttgctggtgtcgatgttggaccatgcccacgggccgttgatggtcatcgctgtttcgcctttattaaaggcagcttctgcgatggagtaatcggtgtctgcattcatgtgtttgtttttaatcaggtcaaccaggaaggtcagacccgctttcgcgccagcgttatccacgcccacgtctttaatgtcgtacttgccgttttcatacttgaacgcataacccccgtcagcagcaatcagcggccaggtgaagtacggttcttgcaggttgaacatcagcgcgctcttacctttcgctttcagttctttatccagcgccgggatctcttcccaggtttttggcgggttcggcagcagatctttgttataaatcagcgataacgcttcaacagcgatcgggtaagcaatcagcttgccgttgtaacgtacggcatcccaggtaaacggatacagcttgtcctggaacgctttgtccggggtgatttcagccaacaggccagattgagcgtagccaccaaagcggtcgtgtgcccagaagataatgtcagggccatcgccagttgccgcaacctgtgggaatttctcttccagtttatccggatgctcaacggtgactttaattccggtatctttctcgaatttcttaccgacttcagcgagaccgttatagcctttatcgccgttaatccagattaccagtttaccttcttcgattttcatatgtatatctccttcttaaagttaaacaaaattatttctagaggggaattgttatccgctcacaattcccctatagtgagtcgtattaatttcgcgggatcgagatcgatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaaattattgaagcatttatcagggttattgtctcatgagcggatacatatttgaatgtatttagaaaaataaacaaataggggttccgcgcacatttccccgaaaagtgccacctaaattgtaagcgttaatgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 4 - DNA, 1224 bp, Artificial Sequence
  Description of Artificial Sequence Synthetic pMCSG19 nucleotide sequence
atgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcaaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgatcacaagtttgtacaaaaaagcaggctcggaaaccgtgcgtttccagtctcaccaccatcatcatcactcttctggcgtggatctgggtaccgagaacctgtacttccaatccaat

SEQ ID NO: 5 - DNA, 24 bp, Artificial Sequence
  Description of Artificial Sequence Synthetic primer
ttaaacatatgaaatcgaagaagg

SEQ ID NO: 6 - DNA, 37 bp, Artificial Sequence
  Description of Artificial Sequence Synthetic primer
ttataggatccacgccagaagagtgatgatgatggtg

SEQ ID NO: 7 - DNA, 18 bp, Artificial Sequence
  Description of Artificial Sequence Synthetic primer
tacttccaatccaatgcn

SEQ ID NO: 8 - DNA, 16 bp, Artificial Sequence
  Description of Artificial Sequence Synthetic primer
ttatccacttccaatg

SEQ ID NO: 9 - protein, 5 aa, Unknown Organism
  Description of Unknown Organism Enterokinase recognition sequence
DDDDK

SEQ ID NO: 10 - protein, 6 aa, Unknown Organism
  Description of Unknown Organism Thrombin recognition sequence
LVPRGS

SEQ ID NO: 11 - protein, 7 aa, Unknown Organism
  Description of Unknown Organism TEV protease recognition sequence
EXXYXQS

SEQ ID NO: 12 - protein, 8 aa, Unknown Organism
  Description of Unknown Organism PreScission protease recognition sequence
LEVLFQGP

SEQ ID NO: 13 - protein, 7 aa, Unknown Organism
  Description of Unknown Organism TVMV protease recognition sequence
ETVRFES

SEQ ID NO: 14 - protein, 6 aa, Artificial Sequence
  Description of Artificial Sequence Synthetic 6xHis tag
HHHHHH